(19) United States
(12) Patent Application Publication
(10) Pub. No.: US 2026/0066058 A1
(43) Pub. Date: Mar. 5, 2026

(54) NON-EQUILIBRIUM CHIMERIC SWITCHING FOR ESTIMATING FREE ENERGY DIFFERENCES

(71) Applicant: SB Technology, Inc., Tarrytown, NY (US)

(72) Inventor: Mary Pitman, Irvine, CA (US)

(21) Appl. No.: 19/312,251

(22) Filed: Aug. 27, 2025

Related U.S. Application Data
(60) Provisional application No. 63/690,522, filed on Sep. 4, 2024.

(51) Int. Cl. G16C 20/30; G16C 10/00

(52) U.S. Cl. CPC G16C 20/30; G16C 10/00

(57) ABSTRACT

Methods, systems, and apparatus, including computer programs encoded on a computer storage medium, for estimating free energy differences via non-equilibrium chimeric switching. In one aspect, a method performed by one or more computers is described, the method including: receiving an input specifying a respective set of thermodynamic parameters of each of a first and second molecular system; processing the input to generate a Hamiltonian of an alchemical system including the first and second molecular systems; parametrizing the Hamiltonian with an alchemical progress parameter that implements an alchemical transformation between the first and second molecular systems; computing, via non-equilibrium switching along each alchemical path in the alchemical transformation, a respective free energy difference estimator between a corresponding pair of molecular systems connected by the alchemical path; and combining each of the free energy difference estimators to estimate a free energy difference between the first and second molecular systems.

### CROSS-REFERENCE TO RELATED APPLICATIONS

This application claims the benefit and right of priority of U.S. Provisional Patent Application No. 63/690,522, titled “NON-EQUILIBRIUM CHIMERIC SWITCHING FOR ESTIMATING FREE ENERGY DIFFERENCES”, filed on Sep. 4, 2024, which is hereby incorporated by reference in its entirety.

### TECHNICAL FIELD

This specification relates generally to methods, systems, and apparatus, including computer programs encoded on a computer storage medium, for estimating free energy differences via non-equilibrium chimeric switching.

### BACKGROUND

Binding free energy differences are related to binding affinities, which characterize the strength of an interaction between a chemical compound and a pharmacological target. Chemical compounds exhibiting high binding affinity are typically more potent, involving lower dosages to exert its therapeutic effect. In silico methods, including high-throughput virtual screening of chemical compound libraries and lead compound optimization, often depend on computational estimates of binding affinity to prioritize drug candidates. As a result, accurate and efficient calculation of binding free energy differences plays a prominent role in molecular design and drug development, from early-stage discovery through to clinical evaluation.

### SUMMARY

This specification describes a free energy prediction system implemented as computer programs on one or more computers in one or more locations that can estimate a free energy difference via non-equilibrium chimeric switching.

The free energy prediction system is configured to estimate a free energy difference between: (a) a first molecular system, and (b) a second molecular system, where the first and second molecular systems have different respective Hamiltonians. To do so, the free energy prediction system performs repeat non-equilibrium switching between the first and second molecular systems and one or more intermediate (“chimeric”) molecular systems, each defined along an alchemical transformation connecting the respective Hamiltonians of the first and second molecular systems. Hence the term “non-equilibrium chimeric switching”. The first and second molecular systems are the physical endpoints of the alchemical transformation, while the intermediate molecular system(s) are the unphysical interior point(s) of the alchemical transformation, corresponding to hybridizations (or superpositions) of the physical endpoints. Among other aspects, the free energy prediction system can use the intermediate molecular system(s) to enhance convergence and improve work overlap of non-equilibrium switching. This can be particularly beneficial when the first and second molecular systems have non-overlapping microstates, e.g., corresponding to physical molecular systems having conformational flexibility, topological changes, and/or non-congeneric molecules.

The free energy prediction system is capable of computing generic free energy difference metrics, including both absolute and relative binding free energies of one or more guest molecules to a target molecular structure, e.g., a ligand to a protein of therapeutic interest. The free energy prediction system can be applied to drug discovery campaigns that demand the high accuracy typically associated with equilibrium free energy perturbation (“FEP”) and thermodynamic integration (“TI”) methods, but with significantly reduced computational time and resources. The combination of high accuracy and computational efficiency is achieved, at least in part, by the improved work overlap of the chimeric intermediate(s), the perfect parallelizability of the non-equilibrium switches, and the ability to implement the non-equilibrium switches at significantly faster rates than equilibrium-based methods, which can be limited by long relaxation times. This feature of faster speed and computational efficiency may be imposed on the free energy prediction system due to cost limitations and/or fast design cycles. In addition, where some methods may fail to deliver adequate results, e.g., due to charged species, poor sampling of the phase-space, and/or convergence limitations, the non-equilibrium approach implemented by the free energy prediction system can provide a lightweight and reliable solution, e.g., by utilizing Crooks' fluctuation-dissipation theorem to reduce variances in exponentially averaged work integrals.

Implementations of the free energy prediction system exploit a non-equilibrium distinct, single, common, or dual topology approach in a single solvent simulation box, facilitating convergence of absolute and relative binding free energy differences between physical molecular systems that would involve exceedingly long simulation times using equilibrium-based methods, e.g., when simulating large, flexible, and/or non-congeneric molecules. Moreover, the non-equilibrium approach implemented by the free energy prediction system does not involve full decoupling of chimeric intermediates, but instead starts and/or ends the non-equilibrium switches from one or more such intermediates. The free energy prediction system can also perform non-equilibrium switching from enhanced (e.g., non-equilibrium) ensembles obtained from molecular simulations employing one or more enhanced sampling techniques, e.g., including importance sampling methods, generalized ensemble methods, or both. Current solutions involve lengthy equilibrium-based simulations that can poorly sample rare events and can take longer to generate decorrelated samples. Hence, current solutions may inefficiently sample from the phase-space and may lead to inaccurate free energy difference predictions.

Particular embodiments of the subject matter described in this specification can be implemented so as to realize one or more of the following advantages.

Binding free energy differences are related to binding affinities, reflecting how strongly a drug (e.g., a ligand or peptide) interacts with a target of therapeutic interest (e.g., a protein or other macromolecule). A high binding affinity usually indicates a potent drug, involving lower dosages to achieve a physiological effect. High-throughput virtual (in silico) drug screening of chemical compound libraries, as well as lead compound and peptide optimization, often relies on binding affinity calculations to identify promising drug candidates. Hence, optimization of binding free energy differences is integral to molecular design and developing safe, effective drugs from initial discovery through clinical trials, e.g., for treatment and detection of diseases.

However, currently available methods for estimating binding free energy differences and binding affinities are, in general, limited. Accurate binding free energy calculations, especially those employing Molecular Dynamics simulations, can involve significant computational resources and time, which reduces their applicability for high-throughput virtual screening. Moreover, ensuring adequate sampling of phase-space in molecular simulations is challenging—inadequate sampling typically leads to inaccurate results. Proper sampling is particularly difficult for large and flexible molecules, as well as applications concerning comparisons between non-congeneric molecules. Presently, equilibrium-based free energy methods, particularly equilibrium-based FEP and TI methods, are the prototypical framework as they can provide suitable accuracy, but are usually intractable for screening large collections of candidate molecules due to the limitations of equilibrium sampling.

In contrast, the free energy prediction system described herein performs non-equilibrium chimeric switching for estimating free energy differences, accommodating high computational speed, efficiency, and accuracy, without compromising any one for another. Moreover, the free energy prediction system can incorporate non-equilibrium chimeric switching with one or more additional techniques, including enhanced sampling and/or dual topology approaches, to overcome known limitations of current state-of-the-art alchemical methods, e.g., slow run times and convergence issues due to long relaxation times and/or poor sampling in equilibrium-based alchemical methods (and other methods that use equilibrium sampling).

Implementations of the free energy prediction system are perfectly parallelizable, where each non-equilibrium switch can be run for a short period of time and is independent of each other non-equilibrium switch. This feature allows for non-equilibrium work values to be collected within seconds or minutes, without efficiency losses due to communication across central processing units (“CPUs”), graphics processing units (“GPUs”), and/or application-specific integrated circuits (“ASICs”), even with a fully parallelized run. Moreover, the independence of the non-equilibrium switches allows for flexibility in the amount of data that can be collected, e.g., where sampling can be terminated based on work overlap criteria and additional sampling can be performed at any later time. Compared to some alchemical methods, molecular simulations of the physical molecular systems output by the free energy prediction system are information rich, e.g., allowing for further analysis and data generation. For example, enhanced sampling of physical molecular systems can provide kinetic data, such as transition states between conformations of interest.

The details of one or more embodiments of the subject matter disclosed in this specification are set forth in the accompanying drawings and the description below. Other features, aspects, and advantages of the subject matter disclosed in this specification will become apparent from the description, the drawings, and the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A is a schematic diagram depicting an example of a free energy prediction system for estimating a free energy difference via non-equilibrium chimeric switching.

FIG. 1B is a schematic diagram depicting an example of a non-equilibrium switch implemented by the free energy prediction system of FIG. 1A.

FIG. 1C is an example plot showing an overlap of probability distributions of work computed by the non-equilibrium switch of FIG. 1B.

FIG. 2A is a schematic diagram of an example “distinct topology” approach for estimating an absolute binding free energy difference via non-equilibrium chimeric switching.

FIG. 2B is a schematic diagram of an example “single topology” approach for estimating a relative binding free energy difference via non-equilibrium chimeric switching.

FIG. 3A is a schematic diagram of an example “common topology” approach for estimating an absolute binding free energy difference via non-equilibrium chimeric switching.

FIG. 3B is a schematic diagram of an example “dual topology” approach for estimating a relative binding free energy difference via non-equilibrium chimeric switching.

FIG. 4A is a flow diagram of an example process for estimating a free energy difference via non-equilibrium chimeric switching.

FIG. 4B is a flow diagram of an example process for computing a free energy difference estimator between a pair of molecular systems in accordance with Crooks' fluctuation-dissipation theorem.

FIG. 5A is a schematic diagram depicting an example of a virtual screening system for virtually screening a set of candidate molecules against a target molecular structure.

FIG. 5B is a schematic diagram depicting another example of a virtual screening system for virtually screening pairs of candidate molecules in a set of candidate molecules against a target molecular structure.

FIG. 6 is a flow diagram of an example process for virtually screening a set of candidate molecules against a target molecular structure.

Like reference numbers and designations in the various drawings indicate like elements.

### DETAILED DESCRIPTION

### I. Introduction

This specification describes methods, systems, and apparatus, including computer programs encoded on a computer storage medium, for estimating free energy differences via non-equilibrium chimeric switching. Implementations of the methods, systems, and apparatus disclosed herein can estimate ligand-protein, protein-protein, and protein-nucleic acid binding free energies (and binding affinities), with applications to a diverse range of scientific fields, including chemistry, molecular physics, physical chemistry, molecular modelling, medicine, biotechnology, and pharmacology.

One notable application of the methods, systems, and apparatus disclosed herein is in the areas of drug discovery, drug screening, drug scoring, and lead compound optimization, particularly virtual (in silico) drug discovery, screening, scoring, and lead compound optimization. Implementations of the methods, systems, and apparatus disclosed herein can virtually screen a large collection of candidate molecules against a target molecular structure of therapeutic interest, e.g., to design a therapeutic drug (or compound) including one or more of the candidate molecules having the highest respective binding affinities to the target molecular structure. The collection of candidate molecules can be specified by a user, can include custom-designed molecules (e.g., molecules designed from a common scaffold), and/or can be retrieved from a chemical or biological database. Examples include the Enamine REAL database, the Chemical Entities of Biological Interest (“ChEBI”) database, the chemical database of the European Molecular Biology Laboratory (“ChEMBL”), the Protein-Ligand Benchmark set, among other related databases.

When compared to existing tools, the methods, systems, and apparatus disclosed herein can accommodate computationally fast, efficient, and high-throughput virtual drug discovery, screening, scoring, and lead compound optimization of a collection of candidate molecules. Implementations of the methods, systems, and apparatus disclosed herein can virtually screen hundreds to thousands of candidate molecules against a target molecular structure on the order of hours or days, while existing tools may take upwards of multiple days to screen a single candidate molecule. For example, the collection of candidate molecules can be a collection of ligands or peptides, e.g., congeneric or non-congeneric ligands or peptides, and the target molecular structure can be a protein associated with one or more disease pathways of a disease, e.g., which is being screened against for treating the disease. As another example, the collection of candidate molecules can be a collection of nucleic acid binding proteins, e.g., transcription factors (“TFs”), and the target molecular structure can be a nucleic acid, e.g., a deoxyribonucleic acid (“DNA”) or a ribonucleic acid (“RNA”), associated with one or more pathological conditions, e.g., which is being screened against for assessing gene regulation of the pathological condition(s).

Implementations of the methods, systems, and apparatus disclosed herein can provide one or more of the following advantages over existing tools:
(i) The methods, systems, and apparatus disclosed herein can estimate free energy differences between two physical molecular systems via an alchemical transformation combined with non-equilibrium switching between the two physical molecular systems and one or more intermediate (“chimeric”) molecular systems, which are the unphysical molecular system(s) of the alchemical transformation. Non-equilibrium switching is, in general, considerably faster than currently available equilibrium-based methods for estimating free energy differences, e.g., equilibrium free energy perturbation (“FEP”) and thermodynamic integration (“TI”) methods, as such methods involve equilibrium sampling which is typically slow and inefficient.
(ii) The methods, systems, and apparatus disclosed herein can allow free energy difference estimations to more rapidly converge without the need for equilibrium sampling, without the need for overlap in the microstates of the two physical molecular systems, and/or without the need for sampling of the gas phase of the intermediate molecular system(s).
(iii) The methods, systems, and apparatus disclosed herein can employ one or more enhanced sampling techniques of both the physical endpoints and the unphysical interior point(s) of the alchemical transformation for improved accuracy of non-equilibrium switching. Current non-equilibrium-based methods sample microstates generated with equilibrium-based Molecular Dynamics or Monte Carlo simulations. However, rare microstates that are undersampled in equilibrium-based Molecular Dynamics and Monte Carlo simulations have been theoretically proven to dominate free energy metrics in non-equilibrium switching. The methods, systems, and apparatus disclosed herein can implement enhanced sampling as an optional technique to generate enhanced (e.g., non-equilibrium) ensembles including such rare microstates.

These features and other features relating to the methods, systems, and apparatus disclosed herein are described in more detail below.

### A. Applications of Free Energy Prediction Systems

Examples of a free energy prediction system 100 are described herein that can estimate a free energy difference 140.a:b between: (a) a first molecular system 110.a, and (b) a second molecular system 110.b. Example implementations of the free energy prediction system 100 for estimating binding free energy differences in drug-target interactions are provided throughout, which are of importance in therapeutic drug discovery. However, the free energy prediction system 100 is not limited to these implementations.

In general, the free energy prediction system 100 can be applied to any physical, chemical, biological, or thermodynamic problem that involves, or is otherwise characterized by, a free energy difference 140.a:b between two molecular systems 110.a and 110.b having different respective Hamiltonians 120.a and 120.b. Hence, the term “molecular system” is used broadly herein and, unless otherwise specified, can refer to any thermodynamic system described by a Hamiltonian on a molecular level. For example, the first 110.a and second 110.b molecular systems can be two molecular systems having different molecular entities, two different poses of a given molecular system, two different phases of a given molecular system, two different states of a given molecular system, and so on.

Non-limiting example applications of the free energy prediction system 100 to various scientific problems are described in the following.

1. Chemical Reactions. The free energy prediction system 100 can be applied to chemical reactions. Examples include, but are not limited to, combination (“synthesis”) reactions, decomposition reactions, single replacement (“displacement”) reactions, double replacement (“metathesis”) reactions, combustion reactions, acid-base reactions, neutralization reactions, precipitation reactions, oxidation reactions, reduction reactions, and redox reactions.

As an example, the free energy prediction system 100 can estimate a free energy difference 140.a:b between: (a) a set of reactants of a chemical reaction, and (b) a set of products of the chemical reaction. Here, the first molecular system 110.a can include the set of reactants of the chemical reaction, and the second molecular system 110.b can include the set of products of the chemical reaction. The free energy difference 140.a.b between the sets of reactants and products of the chemical reaction can be an absolute free energy difference of the chemical reaction. For example, the free energy prediction system 100 can estimate the free energy difference 140.a:b between the sets of reactants and products of the chemical reaction to predict whether the chemical reaction will occur spontaneously.

As another example, the free energy prediction system 100 can estimate a free energy difference 140.a:b between: (a) a first chemical reaction, and (b) a second, different chemical reaction. Here, the first molecular system 110.a can include a set of reactants of the first chemical reaction and a set of products of the second chemical reaction, and the second molecular system 110.b can include a set of products of the first chemical reaction and a set of reactants of the second chemical reaction. The free energy difference 140.a.b between the first and second chemical reactions can be a relative free energy difference including a difference between: (a) an absolute free energy difference of the first chemical reaction, and (b) an absolute free energy difference of the second chemical reaction. For example, the free energy prediction system 100 can estimate the free energy difference 140.a:b between the first and second chemical reactions to predict an equilibrium concentration of a solvent containing the respective sets of reactants and products of the first and second chemical reactions.

2. Catalysis. The free energy prediction system 100 can be applied to catalytic activity (“catalysis”). Examples include, but are not limited to, heterogeneous catalysis, homogenous catalysis, enzymatic catalysis, organocatalysis, photocatalysis, and electrocatalysis.

As an example, the free energy prediction system 100 can estimate a free energy difference 140.a:b between: (a) a set of reactants of a chemical reaction, and (b) a set of products of the chemical reaction, where the chemical reaction is enhanced by a catalyst. Here, the first molecular system 110.a can include the set of reactants of the chemical reaction and the catalyst, and the second molecular system 110.b can include the set of products of the chemical reaction and the catalyst. The free energy difference 140.a.b between the sets of reactants and products of the catalyzed chemical reaction can be an absolute free energy difference of the catalyzed chemical reaction. For example, the free energy prediction system 100 can estimate the free energy difference 140.a:b between the sets of reactants and products of the catalyzed chemical reaction to predict a rate of enhancement of the catalyzed chemical reaction over the uncatalyzed chemical reaction.

3. Protein Folding. The free energy prediction system 100 can be applied to protein folding. Examples include, but are not limited to, model peptides, structural motifs, single-domain proteins, multi-domain proteins, membrane protein folding, disordered-to-ordered protein folding, assisted protein folding, and protein folding diseases.

As an example, the free energy prediction system 100 can estimate a free energy difference 140.a:b between: (a) a first pose of a protein, and (b) a second, different pose of the protein. Here, the first molecular system 110.a can include the protein in the first pose, and the second molecular system 110.b can include the protein in the second pose. The first pose can be a folded (e.g., native) pose of the protein, and the second pose can be an unfolded (e.g., denatured) pose of the protein. For example, the free energy prediction system 100 can estimate the free energy difference 140.a:b between the first and second poses of the protein to predict a folding probability of the protein, e.g., whether the protein will adopt its native conformation under certain conditions.

4. Phase transitions and Critical Points. The free energy prediction system 100 can be applied to phase transitions and critical points. Examples include, but are not limited to, first-order phase transitions, second-order phase transitions, classical phase transitions (e.g., melting, boiling, sublimation, condensation, freezing, and deposition), magnetic phase transitions, superconducting phase transitions, quantum phase transitions, liquid crystal phase transitions, and metal-insulator transitions.

As an example, the free energy prediction system 100 can estimate a free energy difference 140.a:b between: (a) a first phase of a substance involved in a phase transition, and (b) a second, different phase of the substance involved in the phase transition. Here, the first molecular system 110.a can include the substance in the first phase, and the second molecular system 110.b can include the substance in the second phase. For classical phase transitions, the first and second phases of the substance can be one of a solid phase, a liquid phase, or a gas phase. For example, the free energy prediction system 100 can estimate the free energy difference 140.a:b between the first and second phases of the substance to predict an amount of heat transfer involved for melting, boiling, or sublimation of the substance to occur.

5. Electrochemical Cells. The free energy prediction system 100 can be applied to electrochemical cells. Examples include, but are not limited to, galvanic (“voltaic”) cells, electrolytic cells, reference cells, standard cells, bio-electrochemical cells, biological cells, research cells, and specialty cells.

As an example, the free energy prediction system 100 can estimate a free energy difference 140.a:b between: (a) an oxidation reaction involved in a redox reaction, (b) a reduction reaction involved in the redox reaction. Here, the first molecular system 110.a can include a set of reactants of the oxidation reaction and a set of products of the reduction reaction, and the second molecular system 110.b can include a set of products of the oxidation reaction and a set of reactants of the reduction reaction. The free energy difference 140.a.b between the oxidation and reduction reactions can be a relative free energy difference including a difference between: (a) an absolute free energy difference of the oxidation reaction, and (b) an absolute free energy difference of the reduction reaction. For example, the free energy prediction system 100 can estimate the free energy difference 140.a:b between the oxidation and reduction reactions to predict an electromotive force (“EMF”) output by an electrochemical cell implementing the redox reaction.

6. Molecular Interactions and Self-Assembly. The free energy prediction system 100 can be applied to molecular interactions and molecular self-assembly. Examples include, but are not limited to, hydrogen bonding, halogen bonding, Van der Walls forces, electrostatic (“ionic”) interactions, dipole-dipole interactions, hydrophobic interactions, virus capsid formation, amyloid fibrils, and lipid bilayers.

As an example, the free energy prediction system 100 can estimate a free energy difference 140.a:b between: (a) a solvent with a solute, and (b) a solvent without the solute. Here, the first molecular system 110.a can include the solvent having the solute dispersed therein, and the second molecular system 110.b can include the solvent free of the solute. For example, the free energy prediction system 100 can estimate the free energy difference 140.a:b between the solvent with and without the solute to predict a solubility, a hydration number, or a critical micelle concentration (“CMC”) of the solute dispersed in the solvent, e.g., a surfactant dispersed in water.

7. Defect Formation. The free energy prediction system 100 can be applied to defect formation in crystals. Examples include, but are not limited to, point defects, line defects or dislocations, planar or surface defects, bulk or volume defects (e.g., precipitates, voids, cracks, and inclusions), defects in ionic and molecular crystals (e.g., Schottky, Frenkel, and protonic defects), and defects in semiconductors (e.g., donor and acceptor states, traps, and dislocation loops).

As an example, the free energy prediction system 100 can estimate a free energy difference 140.a:b between: (a) a crystal lattice of atoms with one or more defects, and (b) the crystal lattice of atoms without the one or more defects. Here, the first molecular system 110.a can include the crystal lattice of atoms having the defect(s) substituting and/or between the atoms, and the second molecular system 110.b can include the crystal lattice of atoms free of the defect(s), e.g., representing a perfect crystal. The defect(s) can include vacancies of the atoms in the crystal lattice, interstitials between the atoms of the crystal lattice, or other defect types. For example, the free energy prediction system 100 can estimate the free energy difference 140.a:b between the crystal lattice of atoms with and without the defect(s) to predict a defect concentration of a material having the crystal lattice.

8. Biochemical Pathways and Metabolism. The free energy prediction system 100 can be applied to biochemical pathways and metabolism. Examples include, but are not limited to, central energy metabolism, carbohydrate metabolism, lipid metabolism, amino acid metabolism, protein metabolism, nucleotide metabolism, detoxification pathways, redox pathways, signaling pathways, hormone related pathways, and specialized pathways.

As an example, the free energy prediction system 100 can estimate a free energy difference 140.a:b between: (a) a set of reactants involved in an adenosine triphosphate (ATP) hydrolysis reaction, and (b) a set of products involved in the adenosine triphosphate hydrolysis reaction. Here, the first molecular system 110.a can include the ATP suspended in a solvent (e.g., having a given concentration of magnesium cations (Mg²⁺) to stabilize the ATP), and the second molecular system 110.b can include adenosine diphosphate (ADP) and inorganic phosphate (Pᵢ) suspended in the solvent. For example, the free energy prediction system 100 can estimate the free energy difference 140.a:b between the sets of reactants and products of the ATP hydrolysis reaction to predict an amount of chemical energy released within a cell under certain conditions.

As another example, the free energy prediction system 100 can estimate a free energy difference 140.a:b between: (a) a first metabolite involved in a metabolic pathway, and (b) a second, different metabolite involved in the metabolic pathway. Here, the first molecular system 110.a can include the first metabolite and an enzyme, and the second molecular system 110.b can include the second metabolite and the enzyme. The first and second metabolites can be a set of reactants and products, respectively, of a given chemical reaction of the metabolic pathway that is catalyzed by the enzyme. For example, the free energy prediction system 100 can estimate the free energy difference 140.a:b between the first and second metabolites involved in the metabolic pathway to predict a respective amount of chemical energy released or absorbed at each chemical reaction in the metabolic pathway.

9. Surface Adsorption. The free energy prediction system 100 can be applied to surface adsorption. Examples include, but are not limited to, gas adsorption (e.g., on solid surfaces), liquid-solid adsorption, adsorption in catalysis, environmental adsorption, industrial adsorption, surface adsorption in nanoscience, biological adsorption, and biomimetic adsorption.

As an example, the free energy prediction system 100 can estimate a free energy difference 140.a:b between: (a) a surface with one or more molecules adsorbed thereto, and (b) the surface without the molecule(s) adsorbed thereto. Here, the first molecular system 110.a can include the surface and the molecule(s), and the second molecular system 110.b can include the surface isolated from the molecule(s). For example, the free energy prediction system 100 can estimate the free energy difference 140.a:b between the surface with and without the molecule(s) adsorbed thereto to predict an equilibrium coverage of the molecule(s) on the surface and/or an effectiveness of the surface in a catalytic and or filtration process involving the molecule(s).

10. Polymer and Colloidal Science. The free energy prediction system 100 can be applied to polymer and colloidal science. Examples include, but are not limited to, polymer solubility and phase behavior, micellization and self-assembly, colloidal stability and aggregation, gelation and crosslinking, adsorption and surface interactions, and crystallization and ordering.

As an example, the free energy prediction system 100 can estimate a free energy difference 140.a:b between: (i) a polymer blend including a mixture of a set of polymers, and (ii) the unmixed set of polymers. Here, the first molecular system 110.a can include the polymer blend, and the second molecular system 110.b can include each polymer in the set of polymers separated from one another. For example, the free energy prediction system 100 can estimate the free energy difference 140.a:b between the polymer blend and the unmixed polymers to predict a miscibility of the set of polymers in the polymer blend (and the conditions under which phase separation occurs).

As another example, the free energy prediction system 100 can estimate a free energy difference 140.a:b between: (a) a colloid with a stabilizer, and (b) the colloid without the stabilizer. Here, the first molecular system 110.a can include the colloid with the stabilizer, and the second molecular system 110.b can include the colloid without the stabilizer. The stabilizer can be a surfactant, an electrolyte, a polymeric stabilizer, or a pickering stabilizer. For example, the free energy prediction system 100 can estimate the free energy difference 140.a:b between the colloid with and without the stabilizer to predict a stability and/or a dispersibility of particles in the colloid.

11. Cell Membrane Dynamics. The free energy prediction system 100 can be applied to cell membrane dynamics and cell membrane transport. Examples include, but are not limited to, cell membrane structure and motion, passive cell membrane transport (e.g., not involving ATP), active cell membrane transport (e.g., involving ATP), endocytosis, exocytosis, cell membrane protein dynamics, specialized cell membrane transport systems, experimental cell membrane transport systems, and applied cell membrane transport systems.

As an example, the free energy prediction system 100 can estimate a free energy difference 140.a:b between: (a) a particle on a first side of a membrane, and (b) the particle on a second, opposite side of the membrane. Here, the first molecular system 110.a can include a first solvent and a second solvent separated by the membrane, where the particle is suspended in the first solvent, and the second molecular system 110.b can include the first and second solvents separated by the membrane, where the particle is suspended in the second solvent. The particle can be an atom, an ion, or a molecule that is transported across the membrane, and the first and second solvents can have different solute concentrations and/or different charge concentrations across the membrane. For example, the free energy prediction system 100 can estimate the free energy difference 140.a:b between the particle on the first and second sides of the membrane to predict an electrochemical gradient across the membrane.

The abovementioned example applications illustrate the broad applicability of the free energy prediction system 100 in predicting a wide range of thermodynamic phenomena across multiple scientific disciplines.

### B. Alchemical Transformations

The free energy prediction system 100 is an example of a free energy prediction system that performs an alchemical transformation 20.a:b to estimate the free energy difference 140.a:b between the first 110.a and second 110.b molecular systems. The term “alchemical transformation” refers to a computational procedure for transforming the first molecular system 110.a into the second molecular system 110.b, and vice versa. More precisely, the alchemical transformation 20.a:b is a homotopy, i.e., a continuous deformation, between the Hamiltonians (Hₐ) 120.a and (H_{b}) 120.b of the first 110.a and second 110.b molecular systems. Note, a truly “continuous” deformation cannot be implemented numerically beyond machine precision due to rounding in floating point number systems. Thus, the alchemical transformation 20.a:b performed by the free energy prediction system 100 is an approximation to the exact, analytical case outlined below.

Formally, each of the Hamiltonians 120.a and 120.b is a continuous scalar function that maps a microstate (x) in a combined, alchemical phase-space (X) to a total energy (E) in an energy-space (Y), with x∈X and E∈Y. The alchemical phase-space, given as X=Xₐ∪X_{b}, includes a union of the phase-spaces (Xₐ) and (X_{b}) of the first 110.a and second 110.b molecular systems. For a distinct topology approach 200, the two phase-spaces are inequivalent Xₐ≠X_{b} but generally share some elements Xₐ∩X_{b}≠0. For a common topology approach 300, the two phase-spaces are equivalent Xₐ=X_{b}=X.

A homotopy between two continuous scalar functions Hₐ, H_{b}: X→Y is defined by a family of continuous scalar functions:

$${H}_{λ} : X \to Y for λ ∈ [ a , b ] ,$$

such that H(x; a)=Hₐ(x) and H(x; b)=H_{b} (x) for all x∈X, and the map (x, λ)→H(x; λ)=H_{λ}(x) is continuous X×[a, b]→Y. For the distinct topology approach 200, the union of the two phase-spaces implies Hₐ(x)=0 for all x∈X_{b}/Xₐ, and H_{b}(x)=0 for all x∈Xₐ/X_{b}. This family of continuous scalar functions (H_{λ}) is referred to herein as the “alchemical Hamiltonian”. The alchemical Hamiltonian 120.λ can be defined as a Hamiltonian 120.λ of an alchemical system 110.λ that includes, e.g., a union of, the first 110.a and second 110.b molecular systems, as parametrized by the “alchemical progress parameter” (λ).

As the alchemical transformation 20.a:b is a homotopy, the free energy difference 140.a:b between the first 110.a and second 110.b molecular systems is thermodynamically equivalent to a path integral along the alchemical progress parameter. The free energy difference 140.a:b truncates the path integral over a monotonic sequence of points, a<c[1]< . . . <c[n]<b, including one or more interior points, c[1], . . . , c[n], that each define a respective intermediate (“chimeric”) molecular system 110.c as:

$$\begin{matrix}{ΔF}_{a \to b} = {\int }_{a}^{b} - {β}^{- 1} {\partial }_{λ} log {Z}_{λ} d λ \\ = {\int }_{a}^{c [ 1 ]} - {β}^{- 1} {\partial }_{λ} log {Z}_{λ} d λ + … + \\ = Δ {F}_{a \to c [ 1 ]} + … + Δ {F}_{c [ n ] \to b} ,\end{matrix} {\int }_{c [ n ]}^{b} - {β}^{- 1} {\partial }_{λ} log {Z}_{λ} d λ$$

where Z_{λ}=∫exp[−βH_{λ}(x)]dx is the partition function of the alchemical system 110.λ, and β is the inverse temperature. Here, ΔF_{i→j} is the free energy difference 140.i:j between a pair of molecular systems 110.i and 110.j connected by an alchemical path 25.i:j in the alchemical transformation 20.a:b. The alchemical path 25.i:j, given as λ∈[i,j], is itself a homotopy, Hᵢ, Hⱼ: X→Y, between the Hamiltonians (Hᵢ) 120.i and (Hⱼ) 120.j of the pair of molecular systems 110.i and 110.j. Broadly, the free energy prediction system 100 is configured to estimate each free energy difference 140.i:j by executing a respective non-equilibrium switch 135.i:j along each alchemical path 25.i:j, and thereafter combine them to estimate the free energy difference 140.a:b between the first 110.a and second 110.b molecular systems.

### II. Examples of Free Energy Prediction Systems

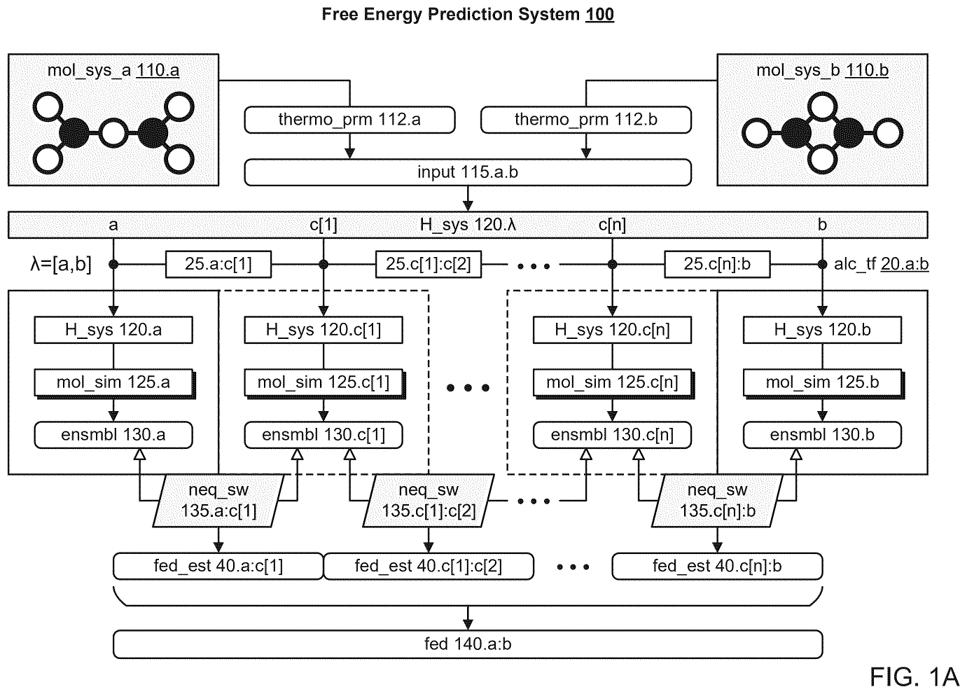

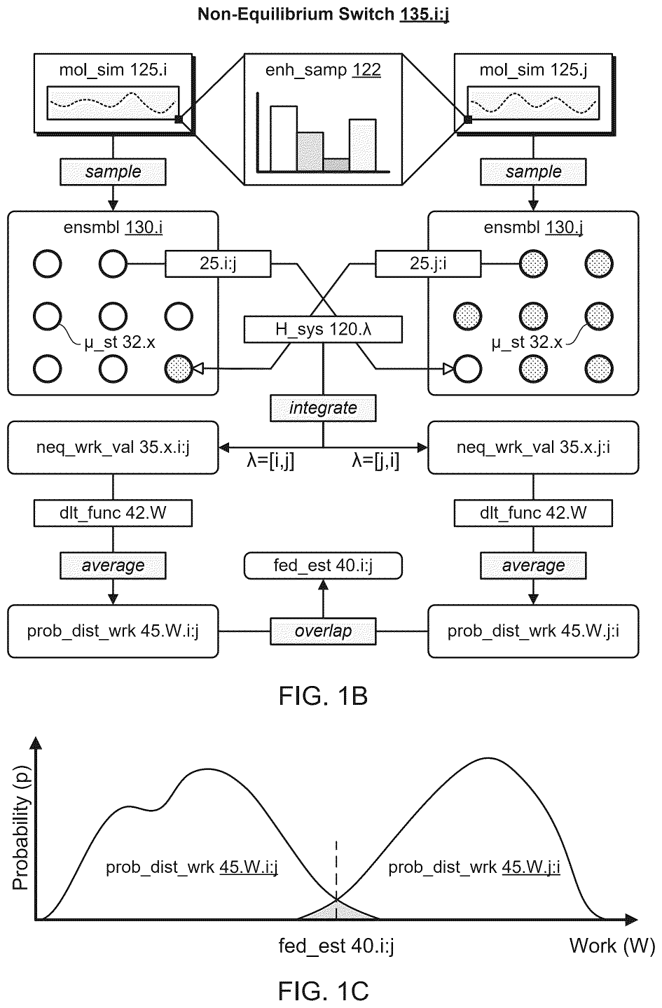

FIG. 1A is a schematic diagram depicting an example of the free energy prediction system 100. The free energy prediction system 100 is an example of a system implemented as computer programs on one or more computers in one or more locations in which the systems, components, and techniques described below are implemented.

In general, the free energy prediction system 100 is configured to estimate a free energy difference 140.a:b between: (a) a first molecular system 110.a, and (b) a second molecular system 110.b. For clarity, the first molecular system 110.a is designated herein as molecular system “a”, and the second molecular system 110.b is designated herein as molecular system “b”. The free energy difference 140.a:b between the first 110.a and second 110.b molecular systems can be any of multiple types of free energy difference metrics, such as a Helmholtz free energy difference, a Gibbs free energy difference, or a Landau free energy difference. For brevity, all such free energy difference metrics will be labelled by ΔF_{a→b} and the distinctions will be made where appropriate.

The free energy difference 140.a:b, given as ΔF_{a→b}=F_{b}−Fₐ, includes a difference between: (a) a free energy (Fₐ) of the first molecular system 110.a, and (b) a free energy (F_{b}) of the second molecular system 110.b. Hence, the reverse process is given as ΔF_{b→a}=−ΔF_{a→b}. The free energies, Fₐ and F_{b}, represent an amount of energy in the first 110.a and second 110.b molecular systems that is available to perform work. If the free energy difference 140.a:b is negative ΔF_{a→b}<0, the second molecular system 110.b is energetically favorable over the first molecular system 110.a, as the second molecular system 110.b involves less free energy. If the free energy difference 140.a:b is positive ΔF_{a→b}>0, the first molecular system 110.a is energetically favorable over the second molecular system 110.b, as the first molecular system 110.a involves less free energy. If the free energy difference 140.a:b is zero ΔF_{a→b}=0, neither the first 110.a nor the second 110.b molecular system is energetically favorable over the other, as both the first 110.a and second 110.b molecular systems involve the same free energy.

In some implementations, the free energy difference 140.a:b measures a driving force of a chemical reaction at a given temperature (T). The balance between a set of reactants and a set of products of the chemical reaction is determined, at least in part, by the free energy difference 140.a:b between the sets of reactants and products of the chemical reaction. The greater the free energy difference 140.a:b, the more the chemical reaction will favor one over the other.

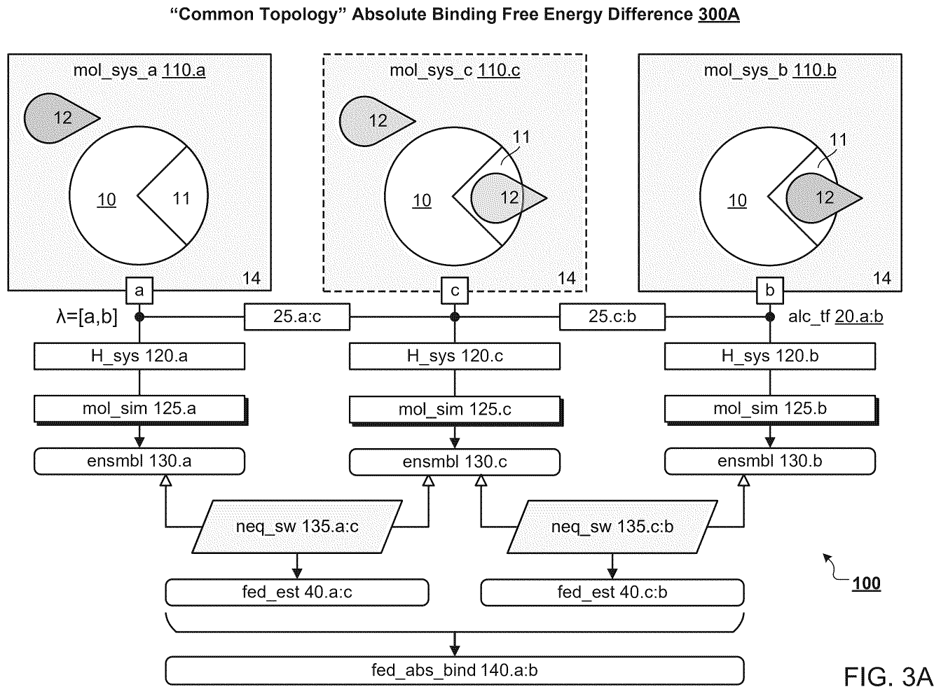

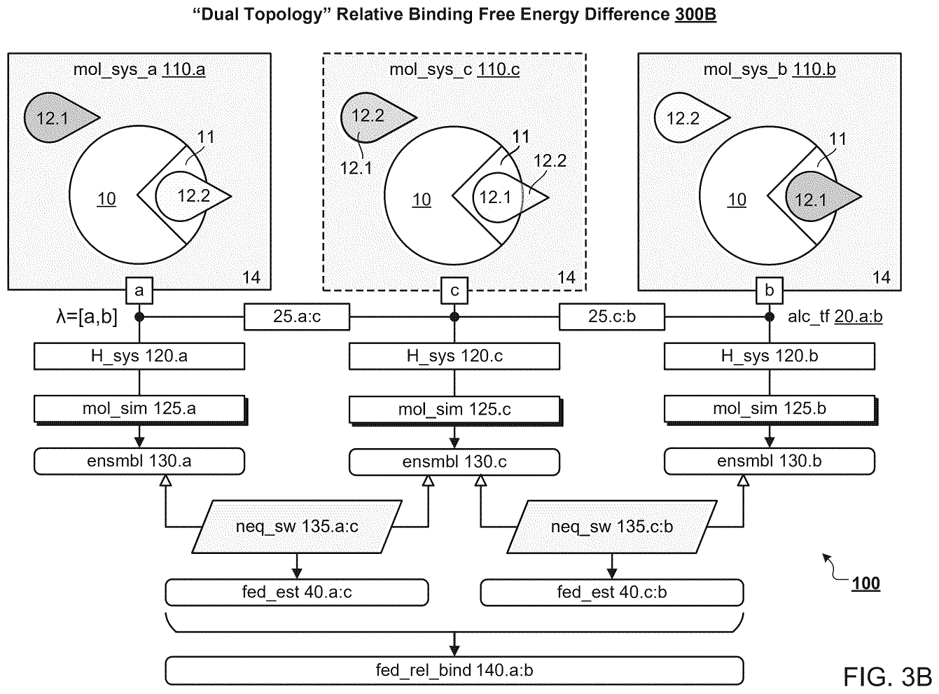

In some implementations, the free energy difference 140.a:b is an absolute free energy difference, ΔF_{a→b}=ΔF⁰, characterizing a chemical reaction at a given temperature. For example, the first molecular system 110.a can include a set of reactants of the chemical reaction, and the second molecular system 110.b can include a set of products of the chemical reaction, e.g., such that the first 110.a and second 110.b molecular systems correspond to opposite sides of a chemical equation describing the chemical reaction. An example of which is shown in the common topology approach 300A of FIG. 3A, corresponding to a binding reaction of a guest molecule (A) 12 to a target molecular structure (B) 10, represented by the chemical equation A+B [symbol] AB.

In some implementations, the free energy difference 140.a:b is a relative free energy difference,

$$Δ {F}_{a \to b} = ΔΔ {F}_{1 , 2}^{0} = Δ {F}_{1}^{0} - Δ {F}_{2}^{0} ,$$

characterizing a difference between two chemical reactions at a given temperature. For example, the first molecular system 110.a can include a set of reactants of a combination of the two chemical reactions, and the second molecular system 110.b can include a set of products of the combined chemical reaction, e.g., such that the first 110.a and second 110.b molecular systems correspond to opposite sides of a chemical equation describing the two chemical reactions together. An example of which is shown in the dual topology approach 300B of FIG. 3B, corresponding to binding reactions of a first guest molecule (A₁) 12.1 and a second guest molecule (A₂) 12.2 to the target molecular structure (B) 10, represented by the chemical equation A₁+A₂B [symbol] A₁B+A₂.

In some implementations, an equilibrium (“disassociation”) constant (K_{D}) between the first 110.a and second 110.b molecular systems is related to the free energy difference 140.a:b as ΔF_{a→b}=−RT log K_{D}, where

$$R \approx 8.314 \frac{J}{K \cdot mol}$$

is the molar gas constant. For a binding reaction,

$$Δ {F}_{a \to b} = Δ {F}_{bind}^{0} ,$$

the disassociation constant provides a measure of a “binding affinity” or “strength” of the binding reaction. In ligand-protein binding for example, a higher binding affinity generally results in a higher occupancy of a protein by a ligand than the case for a lower binding affinity and, therefore, a more potent ligand-protein binding reaction.

To estimate the free energy difference 140.a:b, the free energy prediction system 100 is configured to perform an alchemical transformation 20.a:b between the first 110.a and second 110.b molecular systems. The alchemical transformation 20.a:b refers to a computational process, rather than a physical process, where the first molecular system 110.a is transformed into the second molecular system 110.b, or vise vera, along an unphysical thermodynamic path. In equilibrium-based alchemical methods, e.g., equilibrium FEP and TI methods, an alchemical transformation is performed at rates that maintain a molecular system in equilibrium throughout the alchemical transformation. Here, however, the free energy prediction system 100 performs the alchemical transformation 20.a:b at rates that drive (or maintain) a molecular system 110 out of equilibrium, e.g., allowing faster estimations of the free energy difference 140.a:b compared to equilibrium-based alchemical methods.

To perform the alchemical transformation 20.a:b, the free energy prediction system 100 is configured to generate a Hamiltonian (H_{λ}) 120.λ of an alchemical system 110.λ that includes, e.g., a union of, the first 110.a and second 110.b molecular systems. As mentioned above, the alchemical transformation 20.a:b is unphysical in the sense that it is defined with respect to an alchemical progress parameter (λ), which is an auxiliary, simulation parameter introduced by the free energy prediction system 100. This can be contrasted with a real, physical parameter, such as a strength of an internal force field, a strength of an applied electric field, a distance between a bond, a trapping constant of an optical trap, a size of a Lennard-Jones sphere, and the like. Hence, the alchemical transformation 20.a:b may not represent a physically possible process even though the endpoints of the alchemical transformation 20.a:b are physical. However, since the free energy difference 140.a:b is independent of the thermodynamic path taken between the first 110.a and second 110.b molecular systems, the free energy prediction system 100 can implement the alchemical transformation 20.a:b to estimate the free energy difference 140.a:b irrespective of it being an unphysical process. This affords the free energy prediction system 100 flexibility for accelerating, optimizing, and improving convergence of estimations of the free energy difference 140.a:b, e.g., in implementations when the first 110.a and second 110.b molecular systems have non-overlapping microstates.

For each alchemical path 25.i:j in the alchemical transformation 20.a:b, the free energy prediction system 100 is configured to compute a respective free energy difference estimator (Δ\hat{F}_{i→j}) 40.i:j between the corresponding pair of molecular systems 110.i and 110.j connected by the alchemical path 25.i:j. To compute the free energy difference estimator 40.i:j, the free energy prediction system 100 is configured to execute a non-equilibrium switch 135.i:j along the alchemical path 25.i:j, e.g., in accordance with Jarzynski's non-equilibrium work theorem or Crooks' fluctuation-dissipation theorem. Hence, each non-equilibrium switch 135.i:j starts and/or ends on an intermediate (“chimeric”) molecular system 110.c in the alchemical transformation 20.a:b. For clarity, an intermediate molecular system 110.c is designated herein as molecular system “c”, with the subscript c[1], . . . , c[n] identifying one of the intermediate molecular system(s) 110.c[1] to 110.c[n] in the alchemical transformation 20.a:b.

The free energy prediction system 100 is configured to combine each of the free energy difference estimators 40.a:c[1] to 40.c[n]:b to estimate the free energy difference 140.a:b between the first 110.a and second 110.b molecular systems, e.g., via summation ΔF_{a→b}≈Σ_{i,j}Δ\hat{F}_{i→j}. Here, i→j∈{a→c[1], c[1]→c[2], . . . , c[n−1]→c[n], c[n]→b} runs over the first molecular system 110.a, each intermediate molecular system 110.c[1] to 110.c[n], and the second molecular system 110.b. The notation i→j refers to the deformation between the pair of molecular systems 110.i and 110.j connected by the alchemical path 25.i:j in the alchemical transformation 20.a:b.

The free energy prediction system 100 can be implemented in any appropriate location, e.g., on a user device (e.g., a mobile device), or on one or more computers in a data center, etc. In some implementations, users can interact with the free energy prediction system 100, e.g., by providing a query by way of a user interface, e.g., a graphical user interface (“GUI”), or an application programming interface (“API”). In particular, a user can provide a query including: (i) a request to estimate a free energy difference 140.a:b between a first 110.a and second 110.b molecular system, and (ii) an input 115.a.b specifying a respective set of thermodynamic parameters 112.a and 112.b of each of the first 110.a and second 110.b molecular systems. The free energy prediction system 100 can then process the input 115.a.b, responsive to the request, and provide the free energy difference 140.a:b resulting from the request to the user, e.g., for implementation on a user device of the user, or for storage in a data storage device. In some cases, the free energy difference 140.a:b can transmit the free energy difference 140.a:b to a user device of the user, e.g., by way of a data communication network (e.g., the Internet).

### A. Examples of Molecular Systems and Alchemical Transformations

In general, each of the first 110.a and second 110.b molecular systems is a respective physical molecular system including a respective set of molecular entities that interact with one another, e.g., in accordance with molecular mechanics in a classical mechanics description, or molecular quantum mechanics (“quantum chemistry”) in a quantum mechanics description. For example, the first 110.a and second 110.b molecular systems can each include 10 or more, 20 or more, 30 or more, 40 or more, 50 or more, 100 or more, 150 or more, 200 or more, 250 or more, 500 or more, 750 or more, 1000 or more, 1500 or more, 2000 or more, 2500 or more, or 5000 or more molecular entities.

A “molecular entity” can refer to an atom, a residue, a molecule, an ion, an ion pair, a radical, a radical ion, a complex, a conformer, or any distinguishable entity. Note, the degree of precision involved to describe a molecular entity depends on the context. For example, “hydrogen molecule” may be an adequate definition of a molecular entity in some cases (e.g., molecular mechanics), whereas in other cases (e.g., molecular quantum mechanics), an electronic state, a vibrational state, and/or a nuclear spin state of the hydrogen molecule may also be specified.

In the described examples, each molecular entity in the first 110.a and second 110.b molecular systems is an atom having a respective momentum coordinate and a corresponding position coordinate, and the free energy prediction system 100 models the first 110.a and second 110.b molecular systems in accordance with molecular mechanics. In other examples, one or more of the molecular entities in the first 110.a and/or second 110.b molecular systems can be a group of atoms, e.g., a molecule or a residue of a molecule, that is modeled by the free energy prediction system 100 in accordance with molecular mechanics. In some implementations, the first 110.a and second 110.b molecular systems each include a solvent 14 having their respective sets of molecular entities suspended therein, e.g., corresponding to solute-solvent molecular systems. The solvent 14 can influence dynamics of the molecular entities, e.g., via random collisions and/or frictional drag through the solvent 14. The solvent 14 can provide a thermal bath that maintains the first 110.a and second 110.b molecular systems at a given temperature.

The free energy prediction system 100 is configured to receive an input 115.a.b specifying: (a) a set of thermodynamic parameters ( [symbol] ) 112.a of the first molecular system 110.a, and (b) a set of thermodynamic parameters ( [symbol] ) 112.b of the second molecular system 110.b. For example, the input 115.a.b can be provided by a user of the free energy prediction system 100, an automated system (e.g., a virtual screening system 500), or a combination thereof.

In general, the sets of thermodynamic parameters 112.a and 112.b specify a respective macrostate of each of the first 110.a and second 110.b molecular systems, which allows the free energy prediction system 100 to simulate a respective microstate 32.x of the molecular system 110.a or 110.b consistent with its macrostate. A “microstate” 32.x of a molecular system 110 can refer to a configuration of the molecular system 110 that specifies a respective momentum and position of each atom of the molecular system 110.

The set of thermodynamic parameters 112.a of the first molecular system 110.a can include:

$${𝒜}_{a} = \{ {N}_{a} , {V}_{a} , {P}_{a} , {T}_{a} , {\{ {D}_{al} \}}_{l = 1}^{{N}_{a}} , {\{ {m}_{al} \}}_{l = 1}^{{N}_{a}} , {U}_{a} \} , \qquad ( 1 a )$$

where Nₐ is the total number of atoms in the first molecular system 110.a, Vₐ is the volume of a container enclosing the first molecular system 110.a, Tₐ is the temperature of the first molecular system 110.a, Pₐ is the pressure of the first molecular system 110.a, Dₐₗ is the number of degrees of freedom of the l-th atom in the first molecular system 110.a, mₐₗ is the mass of the l-th atom in the first molecular system 110.a, and Uₐ is an interatomic potential energy between each atom in the first molecular system 110.a. The total number of molecular degrees of freedom (Dₐ) of the first molecular system 110.a is given as

$${D}_{a} = {\sum }_{l - 1}^{{N}_{a}} {D}_{al} ,$$

and the total mass of (Mₐ) of the first molecular system 110.a is given as

$${M}_{a} = {\sum }_{l - 1}^{{N}_{a}} {m}_{al} .$$

The set of thermodynamic parameters 112.a can specify a functional form and a set of force field parameters of the interatomic potential energy of the first molecular system 110.a. In some implementations, the set of force field parameters defines a pose of the first molecular system 110.a, including a respective position and orientation of each atom in the first molecular system 110.a relative to each other atom. Alternatively, the free energy prediction system 100 can initialize the pose of the first molecular system 110.a after receiving the input 115.a.b specifying the set of thermodynamic parameters 112.a. In some implementations, the set of force field parameters includes the effects of a solvent 14 that the set of atoms of the first molecular system 110.a are suspended in.

The set of thermodynamic parameters 112.b of the second molecular system 110.b can include:

$${𝒜}_{b} = \{ {N}_{b} , {V}_{b} , {P}_{b} , {T}_{b} , {\{ {D}_{bl} \}}_{l = 1}^{{N}_{b}} , {\{ {m}_{bl} \}}_{l = 1}^{{N}_{b}} , {U}_{b} \} , \qquad ( 1 b )$$

where N_{b} is the total number of atoms in the second molecular system 110.b, V_{b} is the volume of a container enclosing the second molecular system 110.b, T_{b} is the temperature of the second molecular system 110.b, P_{b} is the pressure of the second molecular system 110.b, D_{bl} is the number of degrees of freedom of the l-th atom in the second molecular system 110.b, m_{bl} is the mass of the l-th atom in the second molecular system 110.b, and U_{b} is an interatomic potential energy between each atom in the second molecular system 110.b. The total number of molecular degrees of freedom (D_{b}) of the second molecular system 110.b is given as

$${D}_{b} = {\sum }_{l = 1}^{{N}_{b}} {D}_{bl} ,$$

and the total mass of (M_{b}) of the second molecular system 110.b is given as

$${M}_{b} = {\sum }_{l = 1}^{{N}_{b}} {m}_{bl} .$$

The set of thermodynamic parameters 112.b can specify a functional form and a set of force field parameters of the interatomic potential energy of the second molecular system 110.b. In some implementations, the set of force field parameters defines a pose of the second molecular system 110.b, including a respective position and orientation of each atom in the second molecular system 110.b relative to each other atom. Alternatively, the free energy prediction system 100 can initialize the pose of the second molecular system 110.b after receiving the input 115.a.b specifying the set of thermodynamic parameters 112.b. In some implementations, the set of force field parameters includes the effects of a solvent 14 that the set of atoms of the second molecular system 110.b are suspended in.

In general, the first 110.a and second 110.b molecular systems are isothermal. That is, the first 110.a and second 110.b molecular systems have the same, given temperature Tₐ=T_{b}=T, e.g., due to thermal contact with a thermal bath (e.g., a solvent 14). For example, if the first 110.a and second 110.b molecular systems represent physical molecular systems in the human body, the given temperature can be equal to T≈310 Kelvin (“K”), e.g., within the normal human body temperature range of 309.5 K to 310.5 K. In some implementations, e.g., for a Helmholtz free energy difference 140.a:b, the first 110.a and second 110.b molecular systems are isothermal-isometric (or isothermal-isochoric). That is, the first 110.a and second 110.b molecular systems have same, given temperature Tₐ=T_{b}=T, and the same, given volume Vₐ=V_{b}=V. In some implementations, e.g., for a Gibbs free energy difference 140.a:b, the first 110.a and second 110.b molecular systems are isothermal-isobaric. That is, the first 110.a and second 110.b molecular systems have same, given temperature Tₐ=T_{b}=T, and the same, given pressure Pₐ=P_{b}=P.

The first 110.a and second 110.b molecular systems are each described by a respective physical Hamiltonian (Hₐ) 120.a and (H_{b}) 120.b, which the free energy prediction system 100 models based on their respective sets of thermodynamic parameters 112.a and 112.b. The Hamiltonians 120.a and 120.b of the first 110.a and second 110.b molecular systems represent the total respective energy for any microstate 32.x of the molecular system 110.a or 110.b.

The Hamiltonian Hₐ=Hₐ(xₐ) 120.a of the first molecular system 110.a is a function of the molecular degrees of freedom (xₐ) of the first molecular system 110.a, which includes a respective momentum (pₐₗ) and corresponding position (qₐₗ) coordinate for each atom in the first molecular system 110.a. That is,

$${x}_{a} = ( {p}_{a} , {q}_{a} ) = {\{ {p}_{al} , {q}_{al} \}}_{l = 1}^{{N}_{a}} .$$

The first molecular system 110.a is three-dimensional, such that the respective momentum and position coordinate of each atom in the first molecular system 110.a includes three components, corresponding to six respective degrees of freedom of the atom Dₐₗ=6. In some implementations, the Hamiltonian 120.a of the first molecular system 110.a is separable in momentum and position coordinates. That is, the Hamiltonian 120.a includes a total kinetic energy (Kₐ) and the interatomic potential energy (Uₐ) of the first molecular system 110.a as:

$${H}_{a} ( {x}_{a} ) = {K}_{a} ( {p}_{a} ) + {U}_{a} ( {q}_{a} ) = {\sum }_{l = 1}^{{N}_{a}} \frac{{p}_{al}^{2}}{2 {m}_{al}} + {U}_{a} ( {q}_{a 1} , {q}_{a 2} , … , {q}_{{aN}_{a}} ) , \qquad ( 2 a )$$

where

$${p}_{al}^{2} = {p}_{al} \cdot {p}_{al}$$

is the squared magnitude of the momentum of the l-th atom in the first molecular system 110.a.

The Hamiltonian H_{b}=H_{b}(x_{b}) 120.b of the second molecular system 110.b is a function of the molecular degrees of freedom (x_{b}) of the second molecular system 110.b, which includes a respective momentum (p_{bl}) and corresponding position (q_{bl}) coordinate for each atom in the second molecular system 110.b. That

$${x}_{b} = ( {p}_{b} , {q}_{b} ) = {\{ {p}_{bl} , {q}_{bl} \}}_{l = 1}^{{N}_{b}} .$$

The second molecular system 110.b is three-dimensional, such that the respective momentum and position coordinate of each atom in the second molecular system 110.b includes three components, corresponding to six respective degrees of freedom for the atom D_{bl}=6. In some implementations, the Hamiltonian 120.b of the second molecular system 110.b is separable in momentum and position coordinates. That is, the Hamiltonian 120.b includes a total kinetic energy (K_{b}) and the interatomic potential energy (U_{b}) of the second molecular system 110.b as:

$${H}_{b} ( {x}_{b} ) = {K}_{b} ( {p}_{b} ) + {U}_{b} ( {q}_{b} ) = {\sum }_{n = 1}^{{N}_{b}} \frac{{p}_{bn}^{2}}{2 {m}_{bn}} + {U}_{b} ( {q}_{b 1} , {q}_{b 2} , … , {q}_{{bN}_{b}} ) . \qquad ( 2 b )$$

where

$${p}_{bl}^{2} = {p}_{bl} \cdot {p}_{bl}$$

is the squared magnitude of the momentum of the l-th atom in the second molecular system 110.b.

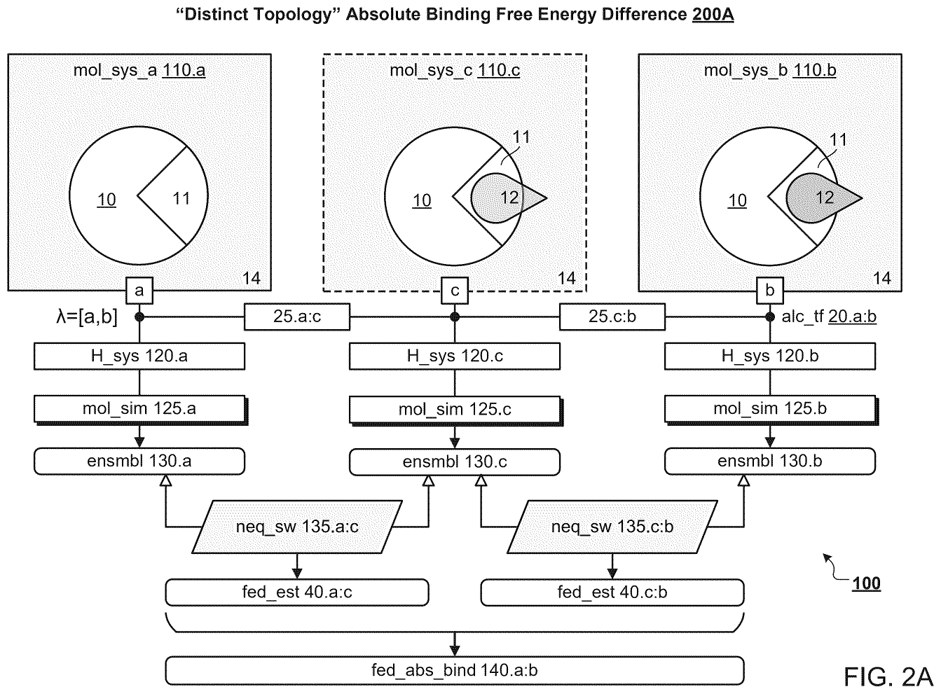

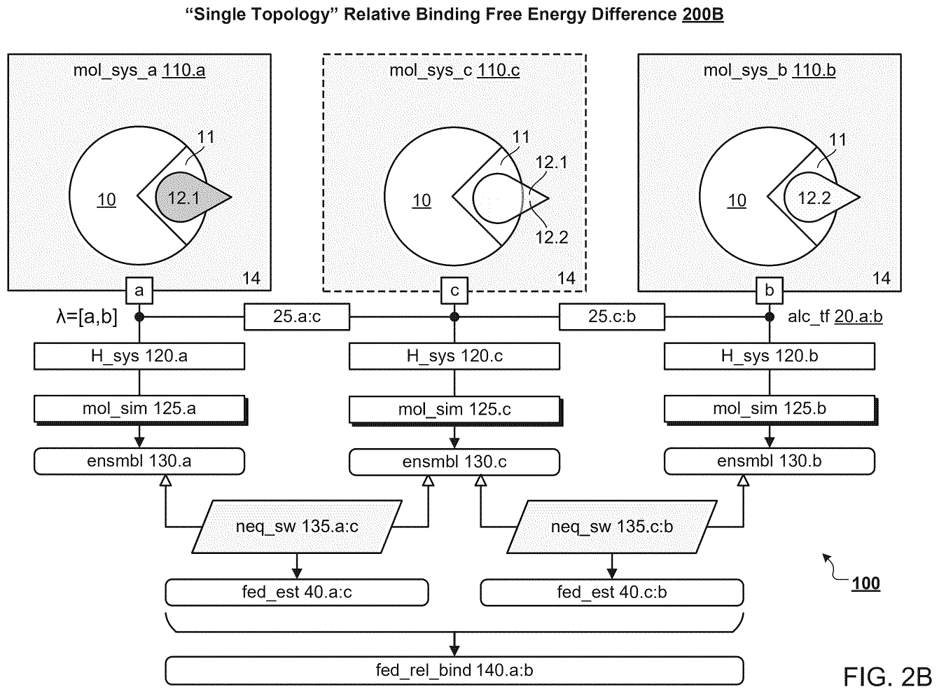

In some implementations, referred to herein as a “distinct topology” approach 200, the first 110.a and second 110.b molecular systems have one or more unique molecular degrees of freedom xₐ≠x_{b}. As shown in FIGS. 2A-2B for example, the first 110.a and second 110.b molecular systems are distinguished by the presence or absence of one or more guest molecules 12 in a single solvent 14 simulation box relative to a target molecular structure 10.

In some implementations, referred to herein as a “common topology” approach 300, the first 110.a and second 110.b molecular systems have the same molecular degrees of freedom xₐ=x_{b}=x. As shown in FIGS. 3A-3B for example, the first 110.a and second 110.b molecular systems are distinguished by rigid translations of the guest molecule(s) 12 in the single solvent 14 simulation box relative to the target molecular structure 10.

More generally, in the common topology approach 300, the first 110.a and second 110.b molecular systems are two respective poses of a given molecular system. Thus, the Hamiltonians 120.a and 120.b of the first 110.a and second 110.b molecular systems are related by a coordinate transformation Hₐ( [symbol] (x))=H_{b}(x), that transposes between the two different poses as:

$${H}_{a} ( 𝒯 ( x ) ) = {H}_{a} ( [ p , Rq + t ) = K ( p ) + {U}_{a} ( Rq + t ) = K ( p ) + {U}_{b} ( q ) = {H}_{b} ( x ) , \qquad ( 2 c )$$

where R is a rotation matrix that individually rotates each atom, and t is a translation vector that individually translates each atom.

In some implementations, the coordinate transformation ( [symbol] ) can include a respective rigid translation of each of one or more guest molecules 12 relative to a target molecular structure 10, e.g., such that each atom in the guest molecule 12 is collectively translated and the target molecular structure 10 is fixed relative to the guest molecule(s) 12.

In some implementations, the first 110.a and second 110.b systems can each include a given set of individual molecules in different poses. In these cases, the coordinate transformation (T) can include a respective rigid translation and/or a respective rigid rotation of each individual ( [symbol] ) molecule in a subset of the given set of individual molecules. The subset of individual molecules can be a proper subset of the given set of individual molecules, e.g., such that each individual molecule in a complement of the subset of individual molecules is fixed relative to the subset of individual molecules. As another example, the subset of individual molecules can be an improper subset of the given set of individual molecules, e.g., such that each individual molecule in the given set is translated and/or rotated relative to each other individual molecule in the given set.

In general, the interatomic potential energies of the first 110.a and second 110.b molecular systems, Uₐ and U_{b}, as specified by the sets of thermodynamic parameters 112.a and 112.b, can each be written as a series expansion of functional terms, where each functional term is one of: (i) a one-body term that depends on the position of one atom; (ii) a two-body term that depends on the positions of two atoms; (iii) a three-body term that depends on the positions of three atoms; and so on. Considering this, the functional forms of the interatomic potential energies can belong to any of multiple classes, including parametric potentials, non-parametric potentials, and combinations thereof. Examples of which are described below.

Parametric potentials include pair potentials, repulsive potentials, many-body potentials, and force fields. Pair potentials describe the potential energy between two interacting atoms as a function of the distance between them. Examples of pair potentials include the Lennard-Jones (or 12-6) potential, the two center Lennard-Jones potential, the shifted Lennard-Jones truncated & shifted potential, the Lennard-Jones truncated & splined potential, the Morse potential, the Buckingham (or exp-6) potential, the Coulomb potential, the Hard Sphere potential, the Sutherland potential, the Mie potential, the Stockmayer potential, the Yukawa potential, the Morse potential, embedded atom model (“EAM”) potentials, among others. Repulsive potentials describe repulsive interactions between two or more interacting atoms. Repulsive potentials can include potentials such as screened Coulomb potentials, potentials derived from density functional theory (“DFT”), potentials derived from tight-binding theory, among others. Many-body potentials describe the potential energy between three or more interacting atoms. Examples of many-body potentials include the Axilrod-Teller potential, the Stillinger-Weber potential, Bond order potentials, among others. Examples of Bond order potentials include the Tersoff potential, the Environment-Dependent Interatomic Potential (“EDIP”), the Brenner potential, the Finnis-Sinclair potentials, ReaxFF, the second-moment tight-binding potentials, among others.

In the context of chemistry, molecular physics, physical chemistry, and/or molecular modelling, a force field is a computational model used by the free energy prediction system 100 to describe the forces between atoms, between collections of atoms, within molecules, or between molecules, spanning a variety of interatomic potentials. More precisely, a force field refers to the functional form and set of force field parameters used by the free energy prediction system 100 to calculate the interatomic potential energies of the first 110.a and second 110.b molecular systems on the atomistic (e.g., molecular) level. The set of force field parameters for a force field may be derived from classical laboratory experiment data, custom tuning, calculations in quantum mechanics, machine learning, or combinations thereof. For example, the force field may include quantum mechanical and/or relativistic corrections such as electronic polarization, nuclear spin, orbital expansion, orbital contraction, spin-orbit coupling, among other effects. Note, force fields have the same interpretation as force fields in classical physics, with the main difference being that the set of force field parameters for a force field in chemistry describes the energy landscape of the Hamiltonians 120.a and 120.b, on the atomistic level. From a force field, the free energy prediction system 100 can derive the acting forces on each atom as a gradient of the interatomic potential energy with respect to the atom's position coordinates.

Non-parametric potentials may be referred to as “machine learning” potentials. Here, the free energy prediction system 100 can use a trained non-parametric potential that provides a prediction of the interatomic potential energies of the first 110.a and second 110.b molecular systems, e.g., as a function of a respective molecular descriptor for each atom in the first 110.a or second 110.b molecular system. For example, a non-parametric potential can be trained to total energies, forces, and/or stresses obtained from quantum-level calculations, e.g., density functional theory or tight-binding theory. Note, the free energy prediction system 100 may combine parametric potentials with non-parametric potentials to describe known physics such as the screened Coulomb repulsion, or impose physical constraints on the first 110.a and second 110.b molecular systems.

The free energy prediction system 100 is configured to process the input 115.a.b specifying the respective sets of thermodynamic parameters 112.a and 112.b of each of the first 110.a and second 110.b molecular systems to generate the Hamiltonian 120.λ of the alchemical system 110.λ including the first 110.a and second 110.b molecular systems. For brevity, the Hamiltonian 120.λ of the alchemical system 110.λ is also referred to herein as the “alchemical Hamiltonian”. The alchemical Hamiltonian 120.λ, given as H_{λ}=H(x; λ), is a function of the alchemical progress parameter (λ) and the combined molecular degrees of freedom (x) of the first 110.a and second 110.b molecular systems.

In some implementations, the molecular degrees of freedom of the alchemical system 110.λ can be represented as

$$x = ( {x}_{0} , {x}_{a}^{sc} , {x}_{b}^{sc} ) ,$$

where x₀ are the common molecular degrees of freedom for atoms that are common to both the first 110.a and second 110.b molecular systems,

$${x}_{a}^{sc}$$

are the “softcore” molecular degrees of freedom for atoms that are unique to the first molecular system 110.a, and

$${x}_{b}^{sc}$$

are the “softcore” molecular degrees of freedom for atoms that are unique to the second molecular system 110.b. The molecular degrees of freedom of the first 110.a and second 110.b are then given as

$${x}_{a} = ( {x}_{0} , {x}_{a}^{sc} ) and {x}_{b} = ( {x}_{0} , {x}_{b}^{sc} ) ,$$

respectively. Other segmentations and coordinate systems (e.g., generalized coordinates) for the combined molecular degrees of freedom are also feasible such as, for example, assigning unique and/or common molecular degrees of freedom to corresponding groups of atoms or whole molecules, e.g., describing the translational, rotational, and/or vibrational degrees of freedom of a group of atoms or molecule. For the common topology approach 300, the molecular degrees of freedom of the alchemical system 110.λ, the first molecular system 110.a, and the second molecular system 110.b are the same xₐ=x_{b}=x.

The free energy prediction system 100 is configured to parametrize the alchemical Hamiltonian 120.λ with the alchemical progress parameter, given as λ∈[a, b], to implement the alchemical transformation 20.a:b between the first 110.a and second 110.b molecular systems. As shown in FIG. 1A, the alchemical transformation 20.a:b is specified in parameter space by:
a monotonic sequence of points, a<c[1]< . . . <c[n]<b, including:
(a) a first endpoint (a) that parametrizes the alchemical Hamiltonian 120.λ as the Hamiltonian H(x; a)=Hₐ(xₐ) 120.a of the first molecular system 110.a;
(b) a second endpoint (b) that parametrizes the alchemical Hamiltonian 120.1 as the Hamiltonian H(x; b)=H_{b} (x_{b}) 120.b of the second molecular system 110.b; and
(c) one or more interior points, c[1], . . . , c[n], that each parametrize the alchemical Hamiltonian 120.λ as a Hamiltonian H (x; c)=H_{c}(x) 120.c of a respective intermediate molecular system 110.c; and
for each contiguous pair of points (i,j) in the monotonic sequence of points, a respective alchemical path λ∈[i,j]25.i:j connecting the pair of points.

The index i∈{a, c[1], . . . , c[n], b}runs over the first molecular system 110.a, each intermediate molecular system 110.c.[1] to 110.c[n], and the second molecular system 110.b. The notation λ∈[i,j] refers to the forward alchemical path 25.i:j between a contiguous pair of molecular systems 110.i and 110.j. The reverse alchemical path 25.j:i is then defined as λ∈[j, i]. The forward 25.i:j and reverse 25.j:i alchemical paths are collectively termed a “conjugate pair” of alchemical paths 25.i:j and 25.j:i.

The intermediate molecular systems 110.c[1] to 110.c[n] are hybridizations (or superpositions) of the first 110.a and second 110.b molecular systems, with the linear or non-linear weighting between the first 110.a and second 110.b molecular systems governed by the parametrization of the alchemical Hamiltonian 120.1. As mentioned above, an intermediate molecular system 110.c is generally unphysical as the alchemical transformation 20.a:b does not represent a physically possible process. The free energy prediction system 100 can use the intermediate molecular system(s) 110.c to provide, for example, overlapping microstates 32.x when the first 110 and second 110.b molecular systems have different energy landscapes in the Hamiltonians 120.a and 120.b.

In some implementations, the endpoints of the alchemical transformation 20.a:b are chosen by the free energy prediction system 100 as a=0 and b=1, such that the interior point(s) of the alchemical transformation 20.a:b have values between zero and one, 0<c[1]< . . . <c[n]<1. For example, the monotonic sequence of points of the alchemical transformation 20.a:b can be a monotonic sequence of equally spaced points from zero to one,

$$i = a + \frac{i ( b - a )}{n + 1} = {i ( n + 1 )}^{- 1} ,$$

which reduces to

$$c = \frac{1}{2}$$

for the intermediate molecular system 110.c in the case of a single interior point. More generally, the free energy prediction system 100 can use any monotonic sequence of points of the alchemical transformation 20.a:b to specify the intermediate molecular system(s) 110.c[1] to 110.c[n].

In some implementations, the free energy prediction system 100 executes multiple runs to estimate the free energy difference 140.a:b and uses different respective sequences and/or numbers of interior points of the alchemical transformation 20.a:b for each run. In these cases, the free energy prediction system 100 may identify from a previous run that a certain spacing between a pair of points provided high (or at least sufficient) work probability overlap during non-equilibrium switching 135.i.j. The free energy prediction system 100 can then reuse this pair of points for the current run and alter the spacing between one or more different pairs of points and/or add one or more additional interior points where there may have been low work probability overlap.

In some implementations, the Hamiltonian 120.λ of the alchemical system 110.λ is a linear combination of the respective Hamiltonians of the first 110.a and second 110.b molecular systems:

$${H}_{λ} ( x ) = ( 1 - λ ) {H}_{a} ( {x}_{a} ) + λ {H}_{b} ( {x}_{b} ) , \qquad ( 3 )$$

where each coefficient in the linear combination is parametrized by the alchemical progress parameter. In the common topology approach 300, the alchemical Hamiltonian 120.λ has the form H_{λ}(x)=K(p)+U_{λ}(q), where K(p) is the kinetic energy of the alchemical system 110.1 that is the same for each of the first 110.a, second 110.b, and intermediate 110.c molecular systems, and U_{λ}=Uₐ+λ(U_{b}−Uₐ) is an alchemical potential energy parametrized by the alchemical progress parameter, which varies between each of the first 110.a, second 110.b, and intermediate 110.c molecular systems. For example, using the monotonic sequence of equally spaced points of the alchemical transformation 20:i.j and the linear interpolation of the alchemical Hamiltonian 120.λ, the effective interatomic potential energy for each molecular system 110.i is

$${U}_{i} = \frac{( n + 1 - i ) {U}_{a} + {iU}_{b}}{n + 1} ,$$

which reduces to

$${U}_{c} = \frac{{U}_{a} + {U}_{b}}{2}$$

for the intermediate molecular system 110.c in the case of a single interior point.

More generally, the alchemical potential energy (U_{λ}) can be a non-linear function of the interatomic potential energies of the first 110.a and second 110.b molecular systems U_{λ}=Uₐ+W_{λ}(u), where u=U_{b}−Uₐ is a perturbation function between the interatomic potential energies, and W_{λ}(u)=W(u; λ) is an alchemical perturbation function. Here, the alchemical perturbation function is a non-linear function (e.g., a functional) of the perturbation function, parametrized by the alchemical progress parameter, that satisfies W (u; a)=0 and W(u; b)=u. For example, the alchemical perturbation function can include a softplus function, a softmax function, a logistic function, a generalized logistic function, or other appropriate non-linear function.

### B. Examples of Non-Equilibrium Switches

FIG. 1B is a schematic diagram depicting an example of a non-equilibrium switch 135.i:j implemented by the free energy prediction system 100. To implement the non-equilibrium switch 135.i:j, the free energy prediction system 100 is configured to vary the alchemical progress parameter along an alchemical path 25.i:j connecting a contiguous pair of points (i,j) of the alchemical transformation 20.a:b. For the forward alchemical path 25.i:j, this transforms (“switches”) the first 110.i of the pair of molecular systems 110.i and 110.j into the second 110.j of the pair of molecular systems 110.i and 110.j. For the reverse alchemical path 25.j:i, this transforms (“switches”) the second 110.j of the pair of molecular systems 110.i and 110.j into the first 110.i of the pair of molecular systems 110.i and 110.j.

In general, the free energy prediction system 100 executes the non-equilibrium switch 135.i:j at a switching rate \dot{λ}≠0 such that the alchemical system 110.1 has insufficient time to relax into a static (or quasi-static) equilibrium. Here, the overdot represents differentiation with respect to time. In some implementations, the switching rate satisfies

$$\dot{λ} > {t}_{relax}^{- 1} ,$$

where tᵣₑₗₐₓ is a relaxation time of the alchemical system 110.λ. For example, the alchemical system 110.λ can have a relaxation time of about 1 nanosecond or more, 10 nanoseconds or more, 100 nanoseconds or more, 1 microsecond or more, 10 microseconds or more, 100 microseconds or more, 1 millisecond or more, 10 milliseconds or more, or 100 milliseconds or more. Moreover, since the alchemical path 25.i:j terminates on at least one interior point, the free energy prediction system 100 performs the non-equilibrium switching 135.i:j between at least one intermediate (“chimeric”) molecular system 110.c. Hence, the term “non-equilibrium chimeric switching”.

In some implementations, the free energy prediction system 100 uses a constant switching rate of

$$\dot{λ} = {t}_{s}^{- 1} ,$$

where tₛ=|tⱼ−tᵢ| is the switching time. The switching time is the period of time it takes the alchemical progress parameter to traverse the alchemical path 25.i:j from the first point λ(tᵢ)=i to the second point λ(tⱼ)=j, or vice versa for the reverse alchemical path 25.j:i. In some implementations, the switching time is less than the relaxation time of the alchemical system 110.λ, that is, tₛ<tᵣₑₗₐₓ. For example, the free energy prediction system 100 can use a switching time of 100 nanoseconds or less, 10 nanoseconds or less, 1 nanosecond or less, 100 picoseconds or less, 10 picoseconds or less, or 1 picosecond or less. In some implementations, the switching time is zero tₛ=0, such that the alchemical progress parameter traverses the alchemical path 25.i:j is instantaneously.

Further, the free energy prediction system 100 can derive and simulate the time evolution of an arbitrary microstate x=(p, q) 32.x of the alchemical system 110.λ while the alchemical progress parameter traverses the alchemical path 25.i:j. The non-equilibrium switch 135.i:j evolves the microstate 32.x in the first 110.i of the pair of molecular systems 110.i and 110.j into a microstate 32.x in the second 110.j of the pair of molecular systems 110.i and 110.j. The free energy prediction system 100 can derive Hamilton's equations of motion for the microstate 32.x under the alchemical Hamiltonian 120.λ as x={x, H_{λ}}, where {⋅,⋅} is the Poisson bracket, and \dot{x} is the rate of change of the microstate 32.x. In terms of the rate of change of the position and momentum coordinates of the microstate 32.x, this amounts to:

$$\dot{q} = \frac{\partial {H}_{λ}}{\partial p} , \qquad ( 4 a )$$

$$and ,$$

$$\dot{p} = \frac{\partial {H}_{λ}}{\partial q} , \qquad ( 4 b )$$

where the time dependence of the alchemical progress parameter λ=λ(t) is implied. For example, for the alchemical Hamiltonian 120.λ of Eq. (3) in the common topology approach 300, the rate of change of the position coordinates is equal to \dot{q}=M⁻¹p, where M=diag(m₁, . . . , M_{N}) is a diagonal matrix including the respective mass of each atom in the alchemical system 110.λ. The rate of change of the momentum coordinates is equal to \dot{p}=−∂_{q}U_{λ}, corresponding to the resulting force on each atom due to the alchemical potential energy.

Eqs. (4a) and (4b) represent the evolution of the microstate 32.x along the alchemical path 25.i:j in accordance with Hamiltonian dynamics. In other implementations, the free energy prediction system 100 evolves the microstate 32.x along the alchemical path 25.i:j in accordance with isothermal dynamics, e.g., Langevin dynamics. For example, to implement Langevin dynamics, the free energy prediction system 100 can modify the rate of change of the momentum coordinates in Eq. (4b) with a frictional force and an uncorrelated random force that function as a thermostat:

$$\dot{p} = - \frac{\partial {H}_{λ}}{\partial q} - γ p + \sqrt{\frac{2 γ M}{β}} \dot{G} ( t ) , \qquad ( 4 {b}^{′} )$$

where γ is a friction coefficient (e.g., proportional to a viscosity of a solvent 14), G(t) is a collection of independent standard Wiener processes (aka Brownian motion), β⁻¹=k_{B}T is the inverse temperature, and k_{B} is Boltzmann's constant. For reference, a Weiner process is a one-parameter family of Gaussian random variables with expectation values of zero and covariances of [symbol] [G(s)G(t)]=min{s, t}.

The free energy prediction system 100 can evaluate a non-equilibrium work 35.x.i.j performed on the microstate 32.x during the evolving using the following non-equilibrium work integral:

$${W}_{i \to j} ( x ) = {\int }_{{t}_{i}}^{{t}_{j}} \dot{λ} ( t ) {d}_{λ} {H}_{λ} [ {x}^{′} ( t ) ] dt , \qquad ( 4 c )$$

where x′(tᵢ)=x is a trajectory of the microstate (x) 32.x as it evolves between a time interval of t=tᵢ to tⱼ, corresponding to the terminating points of the alchemical path 25.i:j in the forward direction, that is, λ(tᵢ)=i and λ(tⱼ)=j. Here, the term ∂_{λ}H_{λ}, represents an “alchemical force” exerted on the microstate 32.x along the trajectory as the alchemical progress parameter is varied in time. For example, for the alchemical Hamiltonian 120.λ of Eq. (3) in the common topology approach 300, the alchemical force is equivalent to ∂_{λ}H_{λ}=∂_{λ}U_{λ}.

Note, in implementations when the switching time is zero tₛ=0, the trajectory of the microstate 32.x is instantaneous x′(tᵢ)=x′(tⱼ)=x. The non-equilibrium work value 35.x.i:j of Eq. (4c) then amounts to the difference in energies of the microstate 32.x evaluated on the respective Hamiltonians 120.i and 120j of the pair of molecular systems 110.i and 110.j, that is, W_{i→j}(x)=Hⱼ(x)−Hᵢ(x).

Eq. (4c) holds for the forward alchemical path 25.i:j, that is, while the alchemical progress parameter traverses the alchemical path 25.i:j in the forward direction. The free energy prediction system 100 can perform the same procedure for the reverse directed path 25.j:i, that is, while the alchemical progress parameter traverses the alchemical path 25.i:j in the reverse direction. In this case, the free energy prediction system 100 evolves a microstate 32.x in the second 110.j of the pair of molecular systems 110.i and 110.j into a microstate 32.x in the first 110.i of the pair of molecular systems 110.i and 110.j. The free energy prediction system 100 can evaluate a non-equilibrium work 35.x.j:i performed on the microstate 32.x during the evolving using the following (reverse) non-equilibrium work integral:

$${W}_{j \to i} = {\int }_{{t}_{j}}^{{t}_{i}} \dot{λ} ( t ) {d}_{λ} {H}_{λ} [ {x}^{′} ( t ) ] dt , \qquad ( 4 d )$$

where x′(tⱼ)=x is a trajectory of the microstate (x) 32.x as it evolves between a time interval of t=tⱼ to tᵢ, corresponding to the terminating points of the alchemical path 25.i:j in the reverse direction, that is, λ(tⱼ)=j and λ(tᵢ)=i.

In general, the free energy prediction system 100 numerically simulates the trajectory of the microstate 32.x, e.g., in accordance with Hamiltonian dynamics in Eqs. (4a) and (4b) or Langevin dynamics in Eqs. (4a) and (4b′), and computes the non-equilibrium work integrals in Eqs. (4c) and (4d) numerically over the trajectory. In other words, as the free energy prediction system 100 switches the alchemical progress parameter along the alchemical path 25.i:j, both the trajectory of the microstate 32.x and the alchemical progress parameter evolve in discrete timesteps, as the time coordinate is discretized. To determine the trajectory of the microstate 32.x at each timestep, the free energy prediction system 100 can perform a Molecular Dynamics (“MD”) simulation 125.MD.X on the microstate 32.x over each of the timesteps, where the Molecular Dynamics simulation 125.MD.X is derived from the alchemical Hamiltonian 120.1 as the alchemical progress parameter traverses the alchemical path 25.i:j.

The free energy prediction system 100 can use any of multiple types of numerical methods and associated numerical integrators to perform the Molecular Dynamics simulation 125.MD.λ. As one example, the free energy prediction system 100 can implement a Verlet integration method, e.g., the leapfrog Stormer-Verlet integration method, with an appropriate Verlet-style numerical integrator on the Hamilton equations of motion in Eqs. (4a) and (4b) to determine the trajectory of the microstate 32.x at each timestep. As another example, the free energy prediction system 100 can implement a Brunger-Brooks-Karplus (“BBK”) integration method, a van Gunsteren-Berendsen (“GB”) integration method, or a Langevin integration method on the Langevin equations of motion in Eqs. (4a) and (4b′) to determine the trajectory of the microstate 32.x at each timestep. Examples of Langevin integrators include, but are not limited to, the Langevin leapfrog integrator, the Langevin Middle Integrator, the Nose-Hoover Integrator, the Variable Langevin Integrator, and the Langevin Impulse integrator. Further examples of numerical integration methods for Langevin dynamics are provided by Skeel, Robert D., and Jesus A. Izaguirre, “An impulse integrator for Langevin dynamics,” Molecular Physics 100.24 (2002): 3885-3891.

The free energy prediction system 100 can then use any suitable numerical integration technique, e.g., a trapezoidal, a midpoint, or a Simpsons numerical integration technique, to compute each of the non-equilibrium work integrals in Eqs. (4c) and (4d) over the trajectory.

Since each molecular system 110.i is isothermal, e.g., due to thermal contact with a thermal bath (e.g., a solvent 14), the molecular system 110.i is generally in an equilibrium (“canonical”) ensemble. This can be (e.g., formally) defined when the switching rate of the alchemical progress parameter is zero \dot{λ}=0. A molecular system 110.i in an equilibrium ensemble is characterized by its respective equilibrium probability distribution (fᵢ), which is commonly referred to as the Boltzmann distribution. These two terms are used interchangeably herein. The Boltzmann distribution has the following representation in phase-space:

$${f}_{i} ( x ) = \frac{exp [ - β {H}_{i} ( x ) ]}{{Z}_{i}} , \qquad ( 5 )$$

where β⁻¹=k_{B}T is the inverse temperature,

$${k}_{B} \approx 1.3806 \times {10}^{- 23} \frac{J}{K}$$

is Boltzmann's constant, and Zᵢ=∫exp [−βHᵢ(x)]dx is the partition function of the molecular system 110.i. The Boltzmann distribution is proportional to the exponential of the Hamiltonian 120.i of the molecular system 110.i, providing the probability that the molecular system 110.i will be in a microstate 32.x as a function of the microstate 32.x's energy and the temperature of the molecular system 110.i. If the partition function is known, most (or all) measurable thermodynamic quantities of interest are equal to ensemble averages (or expectation values) of an observable (O), which can be defined as a multivariate phase-space integral of the observable over the Boltzmann distribution [symbol] O [symbol] fᵢ=∫O(x)fᵢ(x)dx. For example, the total energy (Ēᵢ) of the molecular system 110.i is the ensemble average of its respective Hamiltonian 120.i over its equilibrium ensemble, Ēᵢ= [symbol] Hᵢ [symbol] _{f}_{ᵢ}=∫Hᵢ(x)fᵢ(x)dx. However, in molecular simulations 125, the Boltzmann distribution is not fully sampled, so relative differences between microstates 32.x are typically used instead.

Here, the thermodynamic quantity of interest is the free energy difference 140.a:b between the first 110.a and second 110.b molecular systems. Jarzynski's non-equilibrium work theorem states that the exponential of a free energy difference 40.i:j between a pair of molecular systems 110.i and 110.j is the ensemble average of an exponential of the non-equilibrium work 35.x.i:j performed on each microstate 32.x in the equilibrium ensemble of molecular system 110.i. For the forward switching process, this is represented concisely as:

$$exp ( - βΔ {F}_{i \to j} ) = {〈 exp ( - β {W}_{i \to j} ) 〉}_{{f}_{i}} = \int exp [ - β {W}_{i \to j} ( x ) ] {f}_{i} ( x ) dx . \qquad ( 6 a )$$

The opposite holds for the reverse switching process exp(−βΔF_{j→i})= [symbol] exp(−βW_{j→i}) [symbol] _{f}_{ⱼ}, corresponding to the ensemble average of an exponential of the non-equilibrium work performed 35.x.j:i on each microstate 32.x in the equilibrium ensemble of molecular system 110.j, with ΔF_{j→i}=−ΔF_{i→j}. Note, the free energy difference 140.i:j from Jarzynski's theorem is equivalently expressed in logarithmic form as:

$$βΔ {F}_{i \to j} = - log [ {〈 exp ( - β {W}_{i \to j} ) 〉}_{{f}_{i}} ] . \qquad ( 6 b )$$

A review of Jarzynski's non-equilibrium work theorem is provided by Jarzynski, Christopher, “Nonequilibrium equality for free energy differences,” Physical Review Letters 78.14 (1997): 2690, and Jarzynski, Christopher, “Equilibrium free-energy differences from nonequilibrium measurements: A master-equation approach,” Physical Review E 56.5 (1997): 5018.

Crooks' fluctuation-dissipation theorem is another form of the non-equilibrium work theorem, relating the free energy difference 40.i:j between the pair of molecular systems 110.i and 110.j to respective probability distributions of work 45.W.i:j and 45.W.j:i performed on the molecular systems 110.i and 110.j. For the forward switching process, the probability distribution of work 45.W.i:j is defined as the ensemble average of a delta function 42.W of the non-equilibrium work 35.x.i:j performed on each microstate 32.x in the equilibrium ensemble of molecular system 110.i:

$${p}_{i \to j} ( W ) = {〈 δ ( W - {W}_{i \to j} ) 〉}_{{f}_{i}} = \int δ [ W - {W}_{i \to j} ( x ) ] {f}_{i} ( x ) dx . \qquad ( 7 a )$$

The opposite holds similarly for the probability distribution of work 45.W.j:i for the reverse switching process p_{j→i}(−W)= [symbol] δ(W+W_{j→i}) [symbol] _{f}_{ⱼ}, corresponding to the ensemble average of a delta function 42.W of the non-equilibrium work 35.x.j:i performed on each microstate 32.x in the equilibrium ensemble of molecular system 110.j. Note, for numerical stability, the free energy prediction system 100 can approximate the delta function 42.W with a function that approaches the delta function 42.W in the limit of some parameter, e.g., a (normalized) Gaussian function or a (normalized) Lorentzian function that approaches the delta function 42.W in the limit as its full-width-half-max (“FWHM”) approaches zero. The ratio of the probability distributions of work 45.W.i:j and 45.W.j:i provides Crooks' fluctuation-dissipation theorem:

$$exp [ - β ( Δ {F}_{i \to j} - W ) ] = \frac{{p}_{i \to j} ( W )}{{p}_{j \to i} ( - W )} . \qquad ( 7 b )$$

As shown in Eq. (6b), the particular value of work W=ΔF_{i→j} that equals the free energy difference 140.i:j between the pair of molecular system 110.i and 110.j has an equal likelihood of being performed on each of the pair of molecular systems 110.i and 110.j. In other words, the free energy difference 140.i:j between the pair of molecular system 110.i and 110.j is the intersection point of the probability distributions of work 45.W.i:j and 45.W.j:i, that is, p_{i→j}(ΔF_{i→j})=p_{j→i}(−ΔF_{i→j})=p_{j→i}(ΔF_{j→i}). The free energy difference 40.i:j from Crooks' theorem is equivalently expressed in logarithmic form as:

$$β ( Δ {F}_{i \to j} - W ) = - log [ \frac{{p}_{i \to j} ( W )}{{p}_{j \to i} ( - W )} ] . \qquad ( 7 c )$$

For example, plotting the right-hand side of Eq. (7c) against the work values yields a line with a slope of −β. This line intersects the work axis at the particular value of work equaling the free energy difference 140.i:j. This is illustrated more explicitly in FIG. 1C, which includes an example plot of the overlap of the probability distributions of work 45.W.i:j and 45.W.j:i for the pair of molecular systems 110.i and 110.j connected by the alchemical path 25.i:j in the alchemical transformation 20.a:b.

Notice too, Crooks' fluctuation-dissipation theorem recovers Jarzynski's non-equilibrium work theorem (Eq. (5)) when integrating over all possible values of the work:

$$exp ( - βΔ {F}_{i \to j} ) = \int {p}_{i \to j} ( W ) exp ( - β W ) dW = {〈 exp ( - β {W}_{i \to j} ) 〉}_{{f}_{i}} . \qquad ( 8 )$$

Hence, the free energy prediction system 100 can employ Jarzynski's non-equilibrium work theorem to determine the free energy difference 140.i:j using the forward or reverse switching process. Alternatively, the free energy prediction system 100 can employ Crook's fluctuation-dissipation theorem to determine the free energy difference 140.i:j using both the forward and reverse switching processes. A review of Crooks' fluctuation-dissipation theorem is provided by Crooks, Gavin E, “Entropy production fluctuation theorem and the nonequilibrium work relation for free energy differences,” Physical Review E 60.3 (1999): 2721, and Sevick, Edith M., et al., “Fluctuation theorems,” Annu. Rev. Phys. Chem. 59 (2008): 603-633.

However, as shown in Eqs. (5)-(8), the ensemble averages are over all microstates 32.x belonging to the phase-space, which is computationally intractable for most molecular systems 110 of interest, e.g., as the multivariate phase-space integrals can involve ˜6N integration variables. Instead, the free energy prediction system 100 approximates the multivariate phase-space integrals by generating ensembles 130 including a finite number of microstates 32.x.

That said, with finite sampling of microstates 32.x, ensemble averages have a measurable uncertainty. The free energy difference 40.i:j in Eqs. (5)-(8) is referred to as a free energy difference estimator 40.i:j. The free energy difference estimator 40.i:j, given as Δ\hat{F}_{i→j}, provides an estimate of the free energy difference 40.i:j between the pair of molecular systems 110.i and 110.j. Moreover, as Jarzynski's and Crooks' theorems involve exponential functions, the free energy difference estimator 40.i:j resulting from each of these theorems can have relatively high variance. The results of exponential averaging (e.g., strongly) depend on the behavior at the tails of a distribution, corresponding to rare microstates 32.x that are typically not as well sampled as the rest of the distribution. The free energy prediction system 100 can alleviate this problem by generating enhanced ensembles 130, e.g., non-equilibrium ensembles, that include such rare microstates 32.x. The details of which are described below.

After computing the respective free energy difference estimator 40.i:j for each alchemical path 25.i:j, the free energy prediction system 100 then combines each of the free energy difference estimators 40.a:c[1] to 40.c[n]:b to estimate the free energy difference 140.a:b between the first 110.a and second 110.b molecular systems. For example, the free energy prediction system 100 can sum each of the free energy difference estimators 40.a:c[1] to 40.c[n]:b to estimate the free energy difference 140.a:b as:

$$Δ {F}_{a \to b} \approx Δ {\hat{F}}_{a \to c [ 1 ]} + Δ {\hat{F}}_{c [ 1 ] \to c [ 2 ]} + … + Δ {\hat{F}}_{c [ n - 1 ] \to c [ n ]} + Δ {\hat{F}}_{c [ n ] \to b} .$$

It is worth mentioning that the free energy prediction system 100 is, in general, perfectly parallelizable as each non-equilibrium switch 135.i:j can be performed in parallel with one another once the respective ensembles 130.a, 130.b, and 130.c of microstates 32.x of each of the first 110.a, second 110.b, and intermediate 110.c molecular systems has been obtained. Particularly, the non-equilibrium work value 35.x performed on each microstate 32.x in an ensemble 130 can be computed in parallel with each other microstate 32.x in each other ensemble 130. This is because the free energy prediction system 100 can simulate the trajectory of a microstate 32.x according to Eqs. (4a)-(4d) independently from one another. For example, in a fully parallelized run, the free energy prediction system 100 may collect the non-equilibrium work values 35 within seconds or less.

### C. Examples of Molecular Simulations

After specifying the respective Hamiltonian 120.i of each molecular system 110.i in the alchemical transformation 20.a:b, the free energy prediction system 100 can be configured to generate a respective ensemble (χᵢ) 130.i of microstates 32.x of the molecular system 110.i using the Hamiltonian 120.i. Particularly, for each molecular system 110.i, the free energy prediction system 100 can perform one or more molecular simulations 125.i that are each derived from the Hamiltonian 120.i of the molecular system 110.i. For example, each molecular simulation 125.i can be a Molecular Dynamics (“MD”) simulation 125.MD.i or a Monte Carlo (“MC”) molecular simulation 125.MC.i derived from the Hamiltonian 120.i of the molecular system 110.i. The ensemble 130.i of microstates 32.x, given as

$${𝒳}_{i} = {\{ {x}_{k} \}}_{k = 1}^{{K}_{i}} ,$$

includes a number (Kᵢ) of microstates 32.x each produced, e.g., generated and/or sampled, from the molecular simulation(s) 125.i. For example, the ensemble 130.i of microstates 32.x can include 10 or more, 20 or more, 30 or more, 40 or more, 50 or more, 100 or more, 150 or more, 200 or more, 250 or more, 500 or more, 750 or more, 1000 or more, 1500 or more, 2000 or more, 2500 or more, or 5000 or more microstates 32.x.

The free energy prediction system 100 can be configured to approximate an ensemble average of an observable, e.g., those defined in Eqs. (5)-(8), over the ensemble 130.i of microstates 32.x as:

$${〈 O 〉}_{{f}_{i}} \approx \frac{1}{{K}_{i}} {\sum }_{k = 1}^{{K}_{i}} O ( {x}_{k} ) . \qquad ( 9 )$$

As shown in Eq. (9), the ensemble average of the observable is a summation of the observable evaluated at each microstate 32.x in the ensemble 130.i, divided by the number of microstates 32.x in the ensemble 130.i. Here, the microstates 32.x of the ensemble 130.i have statistical properties as if they were drawn from the Boltzmann distribution xₖ˜fᵢ(x).

Alternatively, the free energy prediction system 100 can be configured to randomly sample a subset ( [symbol] ) of microstates 32.x from the ensemble 130.i. The subset of microstates 32.x, given as [symbol] χᵢ, includes a number

$$( {K}_{i}^{′} )$$

of microstates 32.x of the ensemble 130.i. The free energy prediction system 100 can average the observable over the subset of microstates 32.x in accordance with Eq. (9), with

$${K}_{i} \to {K}_{i}^{′} .$$

Assuming the subset of microstates 32.x is a proper subset,

$${K}_{i}^{′} < {K}_{i} ,$$

the subset is then a smaller sized ensemble than the original ensemble 130.i generated from the molecular simulation(s) 125.i. The subset of microstates 32.x may also be referred to as a “representative set” of microstates 32.x. For example, the subset of microstates 32.x can include 10 or more, 20 or more, 30 or more, 40 or more, 50 or more, 100 or more, 150 or more, 200 or more, 250 or more, 500 or more, 750 or more, 1000 or more, 1500 or more, 2000 or more, 2500 or more, or 5000 or more microstates 32.x.

The free energy prediction system 100 can use subsets of microstates 32.x of different sizes to optimize speed, accuracy, and/or parallelizability of the non-equilibrium switch 135.i:j, e.g., based on the computational resources available to the free energy prediction system 100. As an example, the free energy prediction system 100 can sample respective subsets of microstates 32.x from the ensembles 130.i and 130.j and perform the non-equilibrium switch 135.i:j on all the microstates 32.x in the subsets in parallel to quickly obtain their associated forward and reverse non-equilibrium work values 35.x.i:j and 35.x.j:i. The free energy prediction system 100 can then employ Crooks' theorem to compute the free energy difference estimator 40.i:j using only the subsets of microstates 32.x, e.g., such that not all the microstates 32.x in the ensembles 130.i and 130.j need be evaluated. In cases when there is insufficient overlap in the probability distributions of work 45.W.i:j and 45.W.j:i to determine the free energy difference estimator 40.i:j, the free energy prediction system 100 can sample additional microstates 32.x from the ensembles 130.i and 130.j and preform the non-equilibrium switch 135.i:j on the additional microstates 32.x in parallel to obtain their associated forward and reverse non-equilibrium work values 35.x.i:j and 35.x.j:i. Upon adding the additional microstates 32.x to the subsets of microstates 32.x, the free energy prediction system 100 can then employ Crooks' theorem to recompute the free energy difference estimator 40.i:j using the updated subsets of microstates 32.x.

### D. Examples of Enhanced Sampling Techniques

In general, the molecular simulation(s) 125.i produce microstates 32.x of the molecular system 110.i in accordance with a simulation probability distribution (we) that is a function, at least implicitly, of the Hamiltonian 120.i of the molecular system 110.i. In some implementations, the simulation probability distribution is the equilibrium probability distribution πᵢ(x)=fᵢ(x). In these cases, the ensemble 130.i of microstates 32.x of the molecular system 110.i is an equilibrium ensemble, as the microstates 32.x are produced from the molecular simulation(s) 125.i in accordance with the Boltzmann distribution. The Boltzmann distribution typically samples microstates 32.x from the phase-space near an initial simulation configuration of the molecular system 110.i. In some implementations, the simulation probability distribution is a non-equilibrium probability distribution πᵢ(x)≠fᵢ(x). Here, the ensemble 130.i of microstates 32.x of the molecular system 110.i is a non-equilibrium ensemble, as the microstates 32.x are produced from the molecular simulation(s) 125.i in accordance with a probability distribution other than the Boltzmann distribution.

In either of these abovementioned implementations, the free energy prediction system 100 can perform the molecular simulation(s) 125.i with one or more “enhanced sampling techniques” 122 to sample rare microstates 32.x. Examples of rare microstates 32.x include, but are not limited to, microstates 32.x having high energies but low probabilities fᵢ(xₖ)≈0, microstates 32.x having low entropies, microstates 32.x having slow transition rates, and microstates 32.x separated from the initial simulation configuration of the molecular system 110.i by energetic barriers. Enhanced sampling techniques 122 can significantly improve the performance of the non-equilibrium switch 135.i:j as rare microstates 32.x have been (e.g., theoretically) proven to dominate the free energy difference estimator 40.i:j.

The free energy prediction system 100 can implement any of multiple types of enhanced sampling techniques 122 to improve sampling of rare microstates 32.x in both Molecular Dynamics simulations 125.MD.i and Monte Carlo molecular simulations 125.MC.i. Examples of enhanced sampling techniques 122 are provided below. Further examples of enhanced sampling techniques 122 are provided in the review article of Hénin, Jérôme, et al. “Enhanced sampling methods for Molecular Dynamics simulations,” arXiv preprint arXiv:2202.04164 (2022).

In general, enhanced sampling techniques 122 can be categorized into three categories: (i) importance sampling methods, (ii) generalized ensemble methods, and (iii) hybrid methods that use both importance sampling and generalized ensemble methods.

Importance sampling methods are a family of methods where the simulation probability distribution is different from the equilibrium probability distribution, i.e., the simulation probability distribution is a non-equilibrium probability distribution, but is defined such that a ratio (“likelihood ratio”) of the simulation and equilibrium probability distributions is known (or can be estimated numerically). Thus, the equilibrium probability distribution and ensemble averages over the equilibrium probability distribution can be calculated from the simulation probability distribution. Typically, this amounts to modifying the Hamiltonian 120.i of the molecular system 110.i in a controlled manner and introducing a “modified” Boltzmann distribution that is a function of the modified Hamiltonian. This can be done to focus sampling on microstates 32.x that contribute more to any ensemble averages of interest, e.g., those defined in Eqs. (5)-(8).

Examples of importance sampling methods include, but are not limited to, umbrella sampling (“localization methods”, “locally enhanced sampling”), adaptive seeding, accelerated Molecular Dynamics, Gaussian-accelerated Molecular Dynamics, non-adaptive biasing potential methods, adaptive biasing potential methods, and adaptive biasing force methods.

Examples of adaptive biasing potential methods include, but are not limited to, hyperdynamics, (“traditional”) metadynamics, and the many variants of metadynamics, e.g., well-tempered metadynamics, well-tempered ensemble metadynamics, multiple walkers metadynamics, parallel-bias metadynamics, infrequent metadynamics, adaptive Gaussian metadynamics, reconnaissance metadynamics, λ-metadynamics, flux-tempered metadynamics, path-metadynamics, funnel metadynamics, algorithms for boundary corrections, transition-tempered metadynamics, metabasin metadynamics, experiment directed metadynamics, ensemble-biased metadynamics, target metadynamics, μ-tempered metadynamics, adaptive-numerical-bias metadynamics, altruistic metadynamics, metadynamics for automatic sampling of quantum property manifolds, metadynamics with scaled hypersphere search, variationally enhanced sampling, the Wang-Landau method, and other adaptive biasing potential methods using the interatomic potential energy as a collective variable.

Examples of adaptive biasing force methods include, but are not limited to, the traditional adaptive biasing force method and its variants, e.g., the multiple-walker adaptive biasing force method and the extended-system adaptive biasing force method.

Generalized ensemble (“extended ensemble”) methods is a family of methods where the simulation probability distribution is the equilibrium probability distribution, and sampling is enhanced by exploiting transitions to other ensembles. Here, the ensembles share the same configuration space, but each has different probability due to differences in their macrostates, e.g., due to different sets of thermodynamic parameters such as temperature, pressure, and/or Hamiltonian parameters.

Generalized ensemble methods include two main approaches: (i) expanded ensemble methods, and (ii) replica exchange methods. Expanded ensemble methods include simulated tempering and simulated scaling. Replica exchange methods include temperature replica exchange (“parallel tempering”) and Hamiltonian Replica Exchange methods. In expanded ensemble simulations, macrostates are explored in a single simulation via a biased random walk in macro state space. On the other hand, in replica exchange simulations, multiple coupled simulations are carried out in parallel, and the simulations periodically exchange microstates with each other. Both methods allow estimation of equilibrium averages of observables.

Hybrid methods include various combinations of these abovementioned importance sampling and generalized ensemble methods. Here, the simulation probability distribution is different from the equilibrium probability distribution, i.e., the simulation probability distribution is a non-equilibrium probability distribution, and sampling is further enhanced by exploiting transitions to other non-equilibrium ensembles.

Examples of hybrid methods include, but are not limited to, a combination of replica exchange and a biasing potential method, replica exchange umbrella sampling, parallel-tempering metadynamics, bias-exchange metadynamics, parallel-tempering in the well-tempered ensemble, replica exchange with collective variable tempering, combinations of metadynamics and other enhanced sampling methods, combinations of metadynamics and structural ensemble determination methods (e.g., parallel-bias metadynamics combined with metainference, parallel-tempering in the well-tempered ensemble combined with experiment directed simulation, metadynamics or bias-exchange metadynamics combined with replica-averaging), and combinations of adaptive biasing force methods and other enhanced sampling methods (e.g., metadynamics combined with extended-system adaptive biasing force and/or Gaussian-accelerated Molecular Dynamics).

Importance Sampling Methods. An example of an importance sampling method that can be implemented in both Molecular Dynamics simulations 125.MD.i and Monte Carlo molecular simulations 125.MC.i is described below.

In general, for an importance sampling method, the free energy prediction system 100 selects the simulation probability distribution such that the likelihood ratio of the equilibrium probability distribution to the simulation probability distribution is known. That is, the reweighting factor

$${w}_{i} ( x ) = \frac{{f}_{i} ( x )}{{π}_{i} ( x )}$$

is known or can be estimated by the free energy prediction system 100 numerically. Reweighting can be effective when the reweighting factor does not vary significantly over the phase-space. Considering this, as one example of an importance sampling method, e.g., a non-adaptive biasing potential method, the free energy prediction system 100 can select a simulation probability distribution of the form:

$${π}_{i} ( x ) = \frac{exp [ - {βℋ}_{i} ( x ) ]}{{𝒵}_{i}} , \qquad ( 10 a )$$

where [symbol] =Hᵢ+U_{i,b} is a modified Hamiltonian of the molecular system 110.i, U_{i,b} is a biasing potential energy (that may be a function of a lower-dimensional collective variable (ξ), e.g., as U_{i,b}(q)=U_{i,b}[ξ(q)]), and [symbol] =∫exp[−β [symbol] (x)]dx is a modified partition function of the molecular system 110.i. The biasing potential energy is introduced by the free energy prediction system 100 to promote rare (“important”) microstates 32.x that, for example, dominate the non-equilibrium switch 135.i:j described in Eqs. (5)-(8). This amounts to a biasing of the Hamiltonian 110.i of the molecular system 110.i. The reweighting factor is then expressed as:

$${w}_{i} ( x ) = \frac{{𝒵}_{i}}{{Z}_{i}} exp [ β {U}_{i , b} ( x ) ] = \frac{exp [ β {U}_{i , b} ( x ) ]}{{〈 exp ( β {U}_{i , b} ) 〉}_{{π}_{i}}} , \qquad ( 10 b )$$

where [symbol] . . . [symbol] _{π}_{ᵢ} is the ensemble average with respect to the simulation probability distribution.

The free energy prediction system 100 can then reweight (or transform) the microstates 32.x produced from the molecular simulation(s) 125.i in accordance with the simulation probability distribution using the reweighting factor. This generates an enhanced ensemble 130.i of microstates 32.x that has statistical properties as if the microstates 32.x were drawn from the Boltzmann distribution, but injected with rare microstates 32.x that are poorly sampled from the Boltzmann distribution itself. For example, the free energy prediction system 100 can compute (or estimate) the means, μ_{π}_{ᵢ}= [symbol] x [symbol] _{π}_{ᵢ} and μ_{f}_{ᵢ}= [symbol] x [symbol] _{f}_{ᵢ}= [symbol] xwᵢ [symbol] _{π}_{ᵢ}, and the variances,

$${σ}_{{π}_{i}}^{2} = {〈 {( x - {μ}_{{π}_{i}} )}^{2} 〉}_{{π}_{i}} and {σ}_{{f}_{i}}^{2} = {〈 {( x - {μ}_{{f}_{i}} )}^{2} 〉}_{{f}_{i}} = {〈 {( {xw}_{i} - {μ}_{{f}_{i}} )}^{2} 〉}_{{π}_{i}} ,$$

of the simulation and equilibrium distributions using the reweighting factor. The free energy prediction system 100 can then perform a linear transformation on the microstates 32.x produced from the molecular simulation(s) 125.i by reweighting each of the microstates as xₖ→(σ_{f}_{ᵢ}/σ_{π}_{ᵢ})(xₖ−μ_{π}_{ᵢ})+μ_{f}_{ᵢ}. Note, the free energy prediction system 100 can use any of multiple types of transformations, e.g., including non-linear transformations, to reweight the microstates 32.x.

Alternatively, the free energy prediction system 100 can reweight (or transform) the ensemble averages of the observables themselves, e.g., those defined in Eq. (9), using the reweighting factor. Similar to above, this generates a reweighted ensemble average [symbol] O [symbol] _{f}_{ᵢ}= [symbol] Owᵢ [symbol] _{π}_{ᵢ} that accounts for the statistical differences between the equilibrium and simulation probability distributions. In this case, the free energy prediction system 100 can reweight the ensemble average by scaling the observable evaluated at each microstate 32.x with the reweighting factor evaluated at the microstate 32.x as O(xₖ)→O(xₖ)wᵢ(xₖ), with xₖ˜πᵢ(x).

As mentioned above, each molecular simulation 125.i performed by the free energy prediction system 100 to generate the ensemble 130.i of microstates can be a Molecular Dynamics simulation or a Monte Carlo molecular simulation, depending on the implementation.

The free energy prediction system 100 can perform a Molecular Dynamics simulation 125.MD.i to generate samples in accordance with the simulation probability distribution by deriving (and simulating) Langevin's equations of motion under the modified Hamiltonian ( [symbol] ) of the molecular system 110.i in Eq. (10a). In terms of the rate of change of the momentum and position coordinates in phase-space, this amounts to:

$$\dot{q} = \frac{\partial {ℋ}_{i}}{\partial p} , \qquad ( 11 a )$$

$$and ,$$

$$\dot{p} = - \frac{\partial {ℋ}_{i}}{\partial q} - γ p + \sqrt{\frac{2 γ M}{β}} \dot{G} ( t ) , \qquad ( 11 b )$$

where, as above, γ is a friction coefficient, and G(t) is a collection of independent standard Wiener processes. Here, each microstate 32.x in the ensemble 130.i corresponds to a respective timestep along a trajectory that evolves according to the Langevin dynamics in Eqs. (11a) and (11b). The free energy prediction system 100 can use any of multiple different numerical integration methods, e.g., any of those described above for Eqs. (4a) and (4b′), to determine the trajectory of the microstate 32.x at each timestep under Langevin dynamics. When implementing an enhanced sampling technique 122 as above, the free energy prediction system 100 can perform the Molecular Dynamics simulation 125.MD.i on shorter time scales than the relaxation time of the molecular system 110.i and, therefore, can recover the statistical properties of the molecular system 110.i from trajectories of limited duration. For example, the Molecular Dynamics simulation 125.MD.i can be on the order of 100 nanoseconds or less, 10 nanoseconds or less, 1 nanosecond or less, 100 picoseconds or less, 10 picoseconds or less, or 1 picosecond or less.

On the other hand, the free energy prediction system 100 can perform a Monte Carlo molecular simulation 125.MC.i to generate samples directly from the simulation probability distribution. Particularly, the free energy prediction system 100 can perform the Metropolis-Hastings algorithm to generate a sequence of samples in the form of a Markov chain. Here, the free energy prediction system 100 uses the Metropolis acceptance criterion to propose a new microstate x′ in the Markov chain, and change from the previous microstate x in the Markov chain to x′ with probability:

$$p ( x \to {x}^{′} ) = min \{ 1 , \frac{{π}_{i} ( {x}^{′} )}{{π}_{i} ( x )} \} = min \{ 1 , \frac{exp [ - {βℋ}_{i} ( {x}^{′} ) ]}{exp [ - {βℋ}_{i} ( x ) ]} \} . \qquad ( 12 )$$

This means that the move always occurs if the energy is lowered (probability is increased), and sometimes occurs if the energy of the next microstate is higher, with the probability given by Eq. (12). Here, each microstate in the ensemble 130.i corresponds to a sample along the Markov chain in the Metropolis-Hastings algorithm.

### E. Examples of Distinct, Single, Common, and Dual Topology Approaches

FIGS. 2A-3B show example implementations of the free energy prediction system 100 for computing absolute and relative binding free energy differences relating to binding reactions. For reference, a binding reaction is an attractive interaction between a guest molecule 12 and a target molecular structure 10, resulting in a stable association in which the guest molecule 12 and the target molecular structure 10 are in close proximity to each other.

Molecules that can participate in molecular binding (“molecular docking”) include, but are not limited to, proteins, peptides, ligands, nucleic acids, carbohydrates, lipids, and small organic molecules. For example, in some implementations, the guest molecule 12 is a ligand, a peptide, or a protein, and the target molecular structure 10 is a protein. The binding reaction of a ligand to a pharmacological target protein, e.g., a target protein associated with one or more disease pathways of a disease, provides the molecular basis for activity of most pharmaceutical compounds. Therefore, predicting ligand-protein binding free energy differences is of importance in therapeutic drug discovery and drug screening for treatment of diseases. In other implementations, the guest molecule 12 can be a nucleic acid binding protein, e.g., a transcription factor (“TF”), and the target molecular structure 10 can be a nucleic acid, e.g., a deoxyribonucleic acid (“DNA”) or a ribonucleic acid (“RNA”). Such implementations are of importance in the regulation of gene expression (“gene regulation”).

While binding free energy differences for binding reactions are of importance in therapeutic drug discovery and gene regulation, the free energy prediction system 100 is not limited to such implementations. As noted above, the free energy prediction system 100 can compute the free energy difference 140.a:b between generic molecular systems 110.a and 110.b corresponding to generic chemical reactions, e.g., combination reactions, decomposition reactions, single-replacement reactions, double-replacement reactions, combustion reactions, acid-base reactions, precipitation reactions, oxidation reactions, reduction reactions, redox reactions, among others.

Referring to FIG. 2A, the free energy prediction system 100 can use a distinct topology approach 200A to estimate an absolute binding free energy difference (“ABFED”) 140 between a guest molecule 12 and a target molecular structure 10 to which the guest molecule 12 binds.

The first molecular system 110.a includes the target molecular structure 10. The second molecular system 110.b includes both the guest molecule 12 and the target molecular structure 10, where the guest molecule 12 is positioned inside a binding site 11 of the target molecular structure 10. The first molecular system 110.a further includes a solvent 14 having the target molecular structure 10 suspended therein. The second molecular system 110.b also includes the solvent 14 having the guest molecule 12 and the target molecular structure 10 suspended therein. The intermediate, chimeric molecular system 110.c is a superposition of the first 110.a and second 110.b molecular systems, corresponding to the guest molecule 12 partially absent and partially inside the binding site 11 of the target molecular structure 10.

The free energy difference 140.a:b between the first 110.a and second 110.b molecular systems is then an

$$ABFED ( Δ {F}_{Bind}^{0} )$$

between the guest molecule 12 and the target molecular structure 10, that is,

$$Δ {F}_{a \to b} = Δ {F}_{Bind}^{0} .$$

Note, if the ABFED 140 is negative

$$Δ {F}_{Bind}^{0} < 0 ,$$

the bound state is energetically favorable over the unbound state.

Referring to FIG. 2B, the free energy prediction system 100 can use a single topology approach 200B to estimate a relative binding free energy difference (or “RBFED”) 140 between two, different guest molecules 12.1 and 12.2 and the target molecular structure 10 to which each of the two guest molecules 12.1 and 12.2 bind. For example, the two guest molecules 12.1 and 12.2 may be congeneric, that is, two congeners that are related to each other by origin, structure, and/or function. In some cases, the first guest molecule 12.1 may be a candidate molecule, e.g., for inclusion in a therapeutic drug, that the free energy prediction system 100 is screening against the target molecular structure 10. The second guest molecule 12.2 may be a known, given molecule, e.g., a “reference molecule” having a similar chemical composition as the candidate molecule and having a known ABFED between the target molecular structure 10.

The first molecular system 110.a includes the first guest molecule 12.1 and the target molecular structure 10, where the first guest molecule 12.1 is positioned inside the binding site 11 of the target molecular structure 10. The second molecular system 110.b includes the second guest molecule 12.2 and the target molecular structure 10, where the second guest molecule 12.2 is positioned inside the binding site 11 of the target molecular structure 10. Again, the first molecular system 110.a further includes a solvent 14 having the first guest molecule 12.1 and the target molecular structure 10 suspended therein. The second molecular system 110.b also includes the solvent 14 having the second guest molecule 12.2 and the target molecular structure 10 suspended therein. The intermediate molecular system 110.c is a superposition of the first 110.a and second 100-b molecular systems, corresponding to both guest molecules 12.1 and 12.2 partially absent and partially inside the binding site 11 of the target molecular structure 10.

The free energy difference 140.a:b between the first 110.a and second 110.b molecular systems is then a

$$RBFED ( ΔΔ {F}_{1 , 2}^{0} )$$

between the two guest molecules 12.1 and 12.2 to the target molecular structure 10, that is,

$$Δ {F}_{a \to b} = ΔΔ {F}_{1 , 2}^{0} = Δ {F}_{Bind , 1}^{0} - Δ {F}_{Bind , 2}^{0} .$$

### Here,

$$Δ {F}_{Bind , 1}^{0}$$

is the ABFED between the first guest molecule 12.1 and the target molecular structure 10, and

$$Δ {F}_{Bind , 2}^{0}$$

is the ABFED between the second guest molecule 12.2 and the target molecular structure 10. If the RBFED 140 is negative

$$ΔΔ {F}_{1 , 2}^{0} < 0 ,$$

the binding reaction of the first guest molecule 12.1 is energetically favorable over the binding reaction of the second guest molecule 12.2. The free energy prediction system 100 may then determine the ABFED of the first guest molecule 12.1 by adding the known ABFED of the second guest molecule 12.2 to the RBFED 140, that is,

$$Δ {F}_{Bind , 1}^{0} = ΔΔ {F}_{1 , 2}^{0} + Δ {F}_{Bind , 2}^{0} .$$

Referring to FIG. 3A, the free energy prediction system 100 can use a common topology approach 300B to estimate an ABFED 140 between a guest molecule 12 and a target molecular structure 10 to which the guest molecule 12 binds.

Here, the first molecular system 110.a is a given molecular system 110 in a first pose, and the second molecular system 110.b is the given molecular system 110 in a second, different pose. The given molecular system 110 includes the guest molecule 12 and the target molecular structure 10. As above, the given molecular system 110 further includes a solvent 14 having the guest molecule 12 and the target molecular structure 10 suspended therein. The first pose of the given molecular system 110, i.e., the first molecular system 110.a, corresponds to the guest molecule 12 positioned outside the binding site 11 of the target molecular structure 10. In the first pose, the guest molecule 12 is positioned far enough away from the binding site 11 that interactions between the guest molecule 12 and the target molecular structure 10 are negligible. The second pose of the given molecular system 110, i.e., the second molecular system 110.b, corresponds to the guest molecule 12 positioned inside the binding site 11 of the target molecular structure 10. The intermediate molecular system 110.c is a superposition of the first 110.a and second 110.b molecular systems, corresponding to the guest molecule 12 partially outside and inside the binding site 11 of the target molecular structure 10. The free energy difference 140,a:b between the first 110.a and second 110.b molecular systems is then an

$$ABFED ( Δ {F}_{Bind}^{0} )$$

between the guest molecule 12 and the target molecular structure 10, that is,

$$Δ {F}_{a \to b} = Δ {F}_{Bind}^{0} .$$

Referring to FIG. 3B, the free energy prediction system 100 can also use a dual topology approach 300B to estimate a RBFED 140 between two, different guest molecules 12.1 and 12.2 and the target molecular structure 10 to which each of the two guest molecules 12.1 and 12.2 bind.

Like FIG. 2B, the first guest molecule 12.1 may be a candidate molecule, and the second guest molecule 12.2 may be a reference molecule. Here, the first molecular system 110.a is a given molecular system 110 in a first pose, and the second molecular system 110.b is the given molecular system 110 in a second, different pose. The given molecular system 110 includes the two guest molecules 12.1 and 12.2 and the target molecular structure 10. The given molecular system 110 further includes a solvent 14 having the two guest molecules 12.1 and 12.2 and the target molecular structure 10 suspended therein.

The first pose of the molecular system 110, i.e., the first molecular system 110.a, corresponds to the first guest molecule 12.1 positioned outside the binding site 11 of the target molecular structure 10, and the second guest molecule 12.2 positioned in the binding site 11 of the target molecular structure 10. In the first pose, the first guest molecule 12.1 is positioned far enough away from the binding site 11 that interactions between the first guest molecule 12.1 and the target molecular structure 10 are negligible. The second pose of the molecular system 110, i.e., the second molecular system 110.b, corresponds to the first guest molecule 12.1 positioned inside the binding site 11 of the target molecular structure 10, and the second guest molecule 12.2 positioned outside the binding site 11 of the target molecular structure 10. In the second pose, the second guest molecule 12.2 is positioned far enough away from the binding site 11 that interactions between the second guest molecule 12.2 and the target molecular structure 10 are negligible. The intermediate molecular system 110.c is a superposition of the first 110.a and second 110.b molecular systems, corresponding to both guest molecules 12.1 and 12.2 partially outside and inside the binding site 11 of the target molecular structure 10. The free energy difference 140.a:b between the first 110.a and second 110.b molecular systems is then a

$$RBFED ( ΔΔ {F}_{1 , 2}^{0} )$$

between the two guest molecules 12.1 and 12.2 to the target molecular structure 10, that is,

$$Δ {F}_{a \to b} = Δ {F}_{1 , 2}^{0} .$$

Note, for the distinct 200A and single 200B topology approaches, there can be atoms in the first molecular system 110.a that have no direct analog in the second molecular system 110.b. These correspond to the softcore molecular degrees of freedom

$$( {x}_{a}^{sc} )$$

of the first molecular system 110.a. During the alchemical transformation 20.a:b, the free energy prediction system 100 can decouple the non-bonded interactions of these “dummy” atoms from the target molecular structure 10, meaning that these atoms in the first molecular system 110.a will remain bonded to the guest molecule 12 but will no longer interact with the target molecular structure 10. Similarly, there can be atoms in the second molecular system 110.b that have no direct analog in the first molecular system 110.a. These correspond to the softcore molecular degrees of freedom

$$( {x}_{b}^{sc} )$$

of the second molecular system 110.b. When transforming the first molecular system 110.a into the second molecular system 110.b, the free energy prediction system 100 initializes these atoms as dummy atoms and then couples them to the target molecular structure 10 and solvent 14 during the alchemical transformation 20.a:b.

Despite the ability to employ dummy atoms, the single topology approach 200B is generally easiest to apply when the atoms and bonds in the guest molecules 12.1 and 12.2 are similar, e.g., when the guest molecules 12.1 and 12.2 are congeneric, are near-superimposable, or when one is a substructure of the other. In cases when the guest molecules 12.1 and 12.2 are dissimilar, e.g., when the guest molecules 12.1 and 12.2 are non-congeneric, the dual topology approach 300B may be preferred as no dummy atoms are involved. Particularly, the dual topology approach 300B can be advantageous over the single topology approach 200B as the total charge of the first 110.a and second 110.b molecular systems is the same and, therefore, constant throughout an alchemical transformation 20.a:b. For example, the dual topology approach 300B facilitates mechanisms such as aromatic ring breaking, bond breaking, scaffold hopping, and/or charge changes between the guest molecules 12.1 and 12.2. This is because, unlike the single topology approach 200B, the alchemical transformation 20.a:b does not involve a “mutation” between the guest molecules 12.1 and 12.2 where dummy atoms are coupled and decoupled from the target molecular structure 10, but instead a “transposition” of the guest molecules 12.1 and 12.2 within the same solvent 14 simulation box. This can provide improved numerical stability for the molecular simulation(s) 125, particularly for Molecular Dynamics simulations which are sensitive to the abovementioned mechanisms. In any case, the free energy prediction system 100 can utilize either the single 200B or dual topology 300B approaches when estimating the RBFED 140, depending on the implementation.

### F. Example Methods for Estimating Free Energy Differences

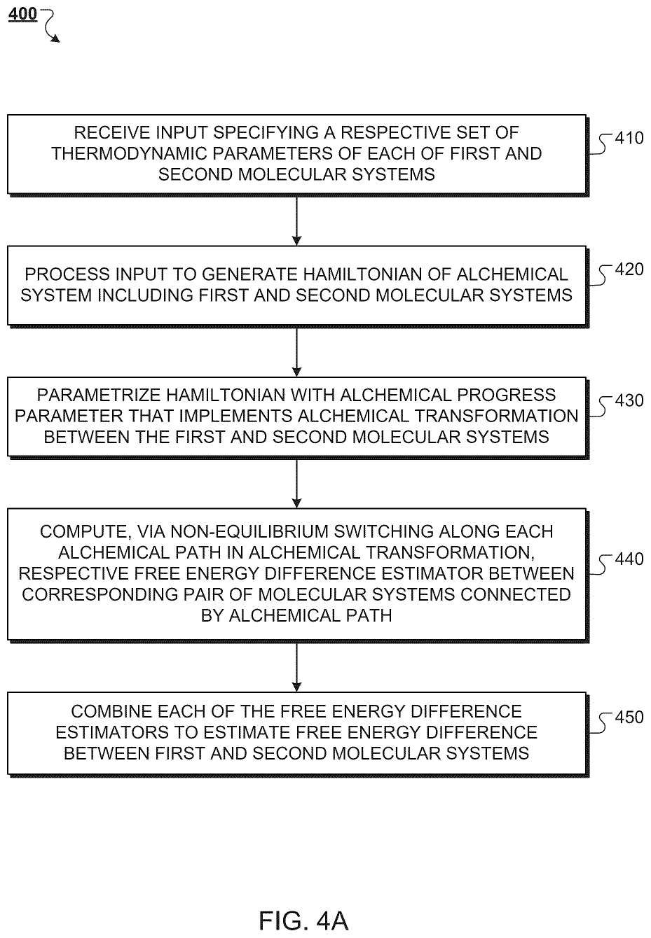

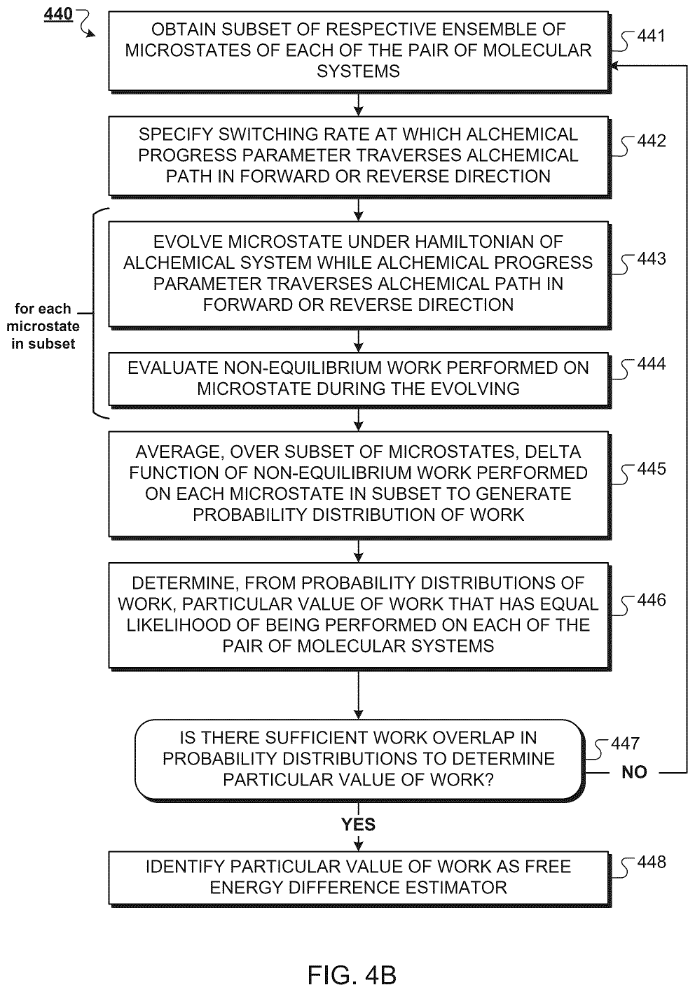

FIG. 4A is a flow diagram of an example process 400 for estimating a free energy difference 140.a:b between a first molecular system 110.a and a second molecular system 110.b. For convenience, the process 400 will be described as being performed by a system of one or more computers located in one or more locations. For example, a free energy prediction system, e.g., the free energy prediction system 100 of FIGS. 1A-1C, appropriately programmed in accordance with this specification, can perform the process 400.

The free energy prediction system 100 receives an input 115.a.b specifying a respective set of thermodynamic parameters 112.a and 112.b of each of the first 110.a and second 110.b molecular systems (410). For example, as explained above, the sets of thermodynamic parameters 112.a and 112.b of the first 110.a and second 110.b molecular systems can include the total number of atoms, the volume of a container (e.g., a solvent 14 box), the temperature, the pressure, the number of degrees of freedom of each atom, the mass of each atom, and the functional form and set of force field parameters of the interatomic potential energy of the first 110.a or second 110.b molecular system.

The free energy prediction system 100 processes the input 115.a.b to generate a Hamiltonian 120.λ of an alchemical system 110.λ including the first 110.a and second 110.b molecular systems (420).

The free energy prediction system 100 parametrizes the Hamiltonian 120.λ with an alchemical progress parameter that implements an alchemical transformation 20.a:b between the first 110.a and second 110.b molecular systems (430). The alchemical transformation 20.a:b is specified by:
a monotonic sequence of points, a<c[1] . . . <c[n]<b, including:
(a) a first endpoint (a) that parametrizes the Hamiltonian 120.λ of the alchemical system 110.λ as a Hamiltonian 120.a of the first molecular system 110.a;
(b) a second endpoint (b) that parametrizes the Hamiltonian 120.λ of the alchemical system 110.λ as a Hamiltonian 120.b of the second molecular system 110.b; and
(c) one or more interior points, c[1], . . . , c[n], that each parametrize the Hamiltonian 120.λ of the alchemical system 110.λ as a Hamiltonian 120.c[1] to 120.c[n] of a respective intermediate molecular system 110.c[1] to 110.c[n]; and
for each contiguous pair of points (i,j) in the monotonic sequence of points, a respective alchemical path 25.i:j connecting the pair of points.

The free energy prediction system 100 computes, via non-equilibrium switching 135.i:j along each alchemical path 25.i:j in the alchemical transformation 20.a:b, a respective free energy difference estimator 40.i:j between the corresponding pair of molecular systems 110.i and 110.j connected by the alchemical path 25.i:j (440).

The free energy prediction system 100 combines each of the free energy difference estimators 40.a:c[1] to 40.c[n]:b to estimate the free energy difference 140.a:b between the first 110.a and second 110.b molecular systems (450). For example, the free energy prediction system 100 can compute a summation or a weighted linear combination of the free energy difference estimators 40.a:c[1] to 40.c[n]:b to estimate the free energy difference 140.a:b.

FIG. 4B is a flow diagram of an example process 440 for computing a free energy difference estimator 40.i:j between a pair of molecular systems 110.i and 110.j connected by an alchemical path 25.i:j in an alchemical transformation 20.a:b. The process 440 involves application of Crooks' fluctuation-dissipation theorem for computing the free energy difference estimator 40.i:j via a non-equilibrium switch 135.i:j, including forward 135.i:j and reverse 135.j:i non-equilibrium switching. However, Jarzynski's non-equilibrium work theorem may also be invoked in other implementations of the process 440. For convenience, the process 440 will be described as being performed by a system of one or more computers located in one or more locations. For example, a free energy difference prediction system, e.g., the free energy prediction system 100 of FIGS. 1A-1C, appropriately programmed in accordance with this specification, can perform the process 440.

The free energy prediction system 100 obtains a subset of a respective ensemble 130.i and 130.j of microstates of each of the pair of molecular systems 110.i and 110.j (441). For example, the free energy prediction system 100 can randomly sample each microstate 32.x in the subsets from the respective ensembles 130.i and 130.j of microstates 32.x of each of the pair of molecular systems 110.i and 110.j. As explained above, to generate an ensemble 130 of microstates of a molecular system 110, that is either a physical endpoint or an unphysical interior point of the alchemical transformation 20.a:b, the free energy prediction system 100 can perform one or more molecular simulations 125 each derived from a Hamiltonian 120 of the molecular system 110. For example, each of the molecular simulation(s) 125 can be a Molecular Dynamics simulation 125.MD or a Monte Carlo molecular simulation 125.MC. In some implementations of the process 440, the free energy prediction system 100 performs the Molecular Dynamics simulation(s) 125.MD or Monte Carlo molecular simulation(s) 125.MC with one or more enhanced sampling techniques 122, e.g., including an importance sampling method, a generalized ensemble method, or both, to generate an enhanced (e.g., non-equilibrium) ensemble 130 of the molecular system 110.

The free energy prediction system 100 specifies a switching rate at which the alchemical progress parameter traverses the alchemical path 25.i:j in a forward or reverse direction (442).

For each microstate 32.x in the subset of the ensemble 130.i of microstates 32.x of a first 110.i of the pair of molecular systems 110.i and 110.j:

The free energy prediction system 100 evolves the microstate 32.x under the Hamiltonian 120.λ of the alchemical system 110.λ while the alchemical progress parameter traverses the alchemical path 25.i:j in the forward direction (443i).

The free energy prediction system 100 evaluates a non-equilibrium work performed on the microstate during the evolving (444i).

The free energy prediction system 100 then averages, over the subset of the ensemble 130.i of microstates 32.x of the first 110.i of the pair of molecular systems 110.i and 110.j, a delta function 42.W of the non-equilibrium work 35.x.i:j performed on each microstate 32.x in the subset to generate a probability distribution of work 45.W.i:j performed on the molecular system 110.i (445i).

For each microstate in the subset of the ensemble 130.j of microstates 32.x of a second 110.j of the pair of molecular systems 110.i and 110.j:

The free energy prediction system 100 evolves the microstate 32.x under the Hamiltonian 120.λ of the alchemical system 110.λ while the alchemical progress parameter traverses the alchemical path 25.i:j in the reverse direction (443j).

The free energy prediction system 100 evaluates a respective non-equilibrium work 35.x.j.i performed on the microstate 32.x during the evolving (444j).

The free energy prediction system 100 then averages, over the subset of the ensemble 130.j of microstates 32.x of the second 110.j of the pair of molecular systems 110.i and 110.j, a delta function 42.W of the non-equilibrium work 35.x.j.i performed on each microstate 32.x in the subset to generate a probability distribution of work 45.W.j.i performed on the molecular system 110.j (445j).

The free energy prediction system 100 determines, from the probability distributions of work 45.W.i.j and 45.W.j.i performed on the pair of molecular systems 110.i and 110.j, a particular value of work that has an equal likelihood of being performed on each of the pair of molecular systems 110.i and 110.j (446). For example, the free energy prediction system 100 can isolate the intersection point of the probability distributions of work 45.W.i.j and 45.W.j.i to determine the particular value of work.

The free energy prediction system 100 determines whether there is sufficient overlap in the probability distributions of work 45.W.i.j and 45.W.j.i to determine the particular value of work (447). If so, the free energy prediction system 100 breaks to operation (448). If not, the free energy prediction system 100 breaks to operation (441) and adds more microstates 32.x to the subsets of the ensembles 130.i and 130.j, e.g., by randomly sampling additional microstates 32.x from the ensembles 130.i and 130.j. The free energy prediction system 100 then performs operations (441)-(444) on the additional microstates 32.x and recomputes the probability distributions of work 45.W.i.j and 45.W.j.i in operation (445).

The free energy prediction system 100 identifies the particular value of work as the free energy difference estimator 40.i:j between the pair of molecular systems 110.i and 110.j (448).

### III. Examples of Virtual Screening Systems

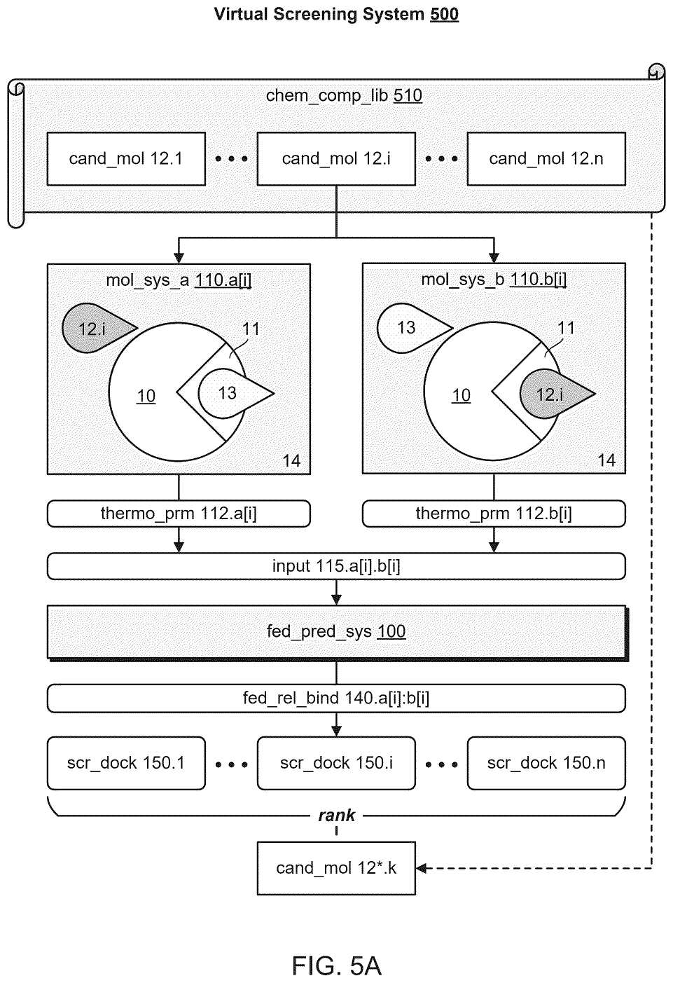

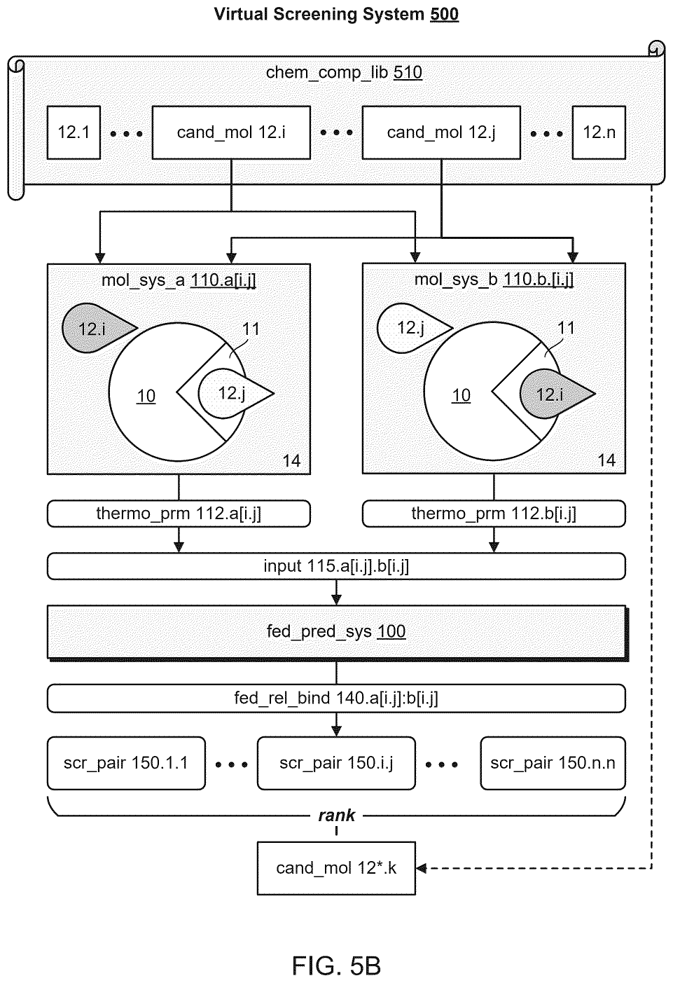

FIGS. 5A and 5B are schematic diagrams depicting an example of a virtual screening system 500 configured to perform virtual drug discovery, virtual drug screening, virtual drug scoring, and/or virtual lead compound optimization, using the free energy prediction system 100. FIG. 5A shows an example of the virtual screening system 500 configured for single molecule screening, and FIG. 5B shows an example of the virtual screening system 500 configured for pair-wise molecule screening. The virtual screening system 500 is an example of a system implemented as computer programs on one or more computers in one or more locations in which the systems, components, and techniques described below are implemented.

The virtual screening system 500 is configured to receive data defining a set of candidate molecules 12.1 to 12.n. The data defining the set of candidate molecules 12.1 to 12.n can be provided by a user of the virtual screening system 500, an automated system, or a combination thereof. In some implementations, a user of the virtual screening system 500 may specify the data defining the set of candidate molecules 12.1 to 12.n, e.g., including molecules or custom-designed molecules. In other implementations, the virtual screening system 500 may retrieve the data from a chemical compound database 510 (or biological database), such as any of those described above. The set of candidate molecules 12.1 to 12.n can include 10 or more, 20 or more, 30 or more, 40 or more, 50 or more, 100 or more, 150 or more, 200 or more, 250 or more, 500 or more, 750 or more, 1000 or more, 1500 or more, 2000 or more, 2500 or more, or 5000 or more candidate molecules 12. The virtual screening system 500 may screen the set of candidate molecules 12.1 to 12.n on the order of hours or days due to the computationally fast non-equilibrium chimeric switching performed by the free energy prediction system 100.

The virtual screening system 500 is configured to generate a set of scores 150.1 to 150.n for the set of candidate molecules 12.1 to 12.n, including a respective score 150.i for each candidate molecule 12.i in the set of candidate molecules 12.1 to 12.n. Particularly, using the free energy prediction system 100, the virtual screening system 500 screens each candidate molecule 12.i against a target molecular structure 10 and determines the respective score 150.i for the candidate molecule 12.i based on a respective free energy difference 140.a[i]:b[i] estimated by the free energy prediction system 100. For example, the set of scores 150.1 to 150.n may be a set of docking scores that characterizes how well the set of candidate molecules 12.1 to 12.n binds to the target molecular structure 10.

In some implementations, the target molecular structure 10 may be associated with one or more disease pathways of a disease. The virtual screening system 500 can screen the set of candidate molecules 12.1 to 12.n against the target molecular structure 10 to aid in designing a therapeutic drug for treating the disease, particularly, for selecting a subset of the candidate molecules 12 for inclusion in the therapeutic drug. For example, each candidate molecule 12.i may be a ligand or a peptide, and the target molecular structure 10 may be a protein. As another example, each candidate molecule 12.i may be a nucleic acid binding protein, and the target molecular structure 10 may be a nucleic acid (e.g., a DNA or an RNA). Here, each candidate molecule 12.i in the set of candidate molecules 12.1 to 12.n is screened against the target molecular structure 10 alongside a reference molecule 13, e.g., having a known ABFED between the target molecular structure 10. For example, in some implementations, the reference molecule 13 and each of the candidate molecules 12.1 through 12.i may be congeneric.

In some implementations, the reference molecule 13 is another one of the candidate molecules 12.j in the set of candidate molecules 12.1 to 12.n, e.g., see FIG. 5B. In these cases, each score 150.i is a pair-wise score 150.i.j that characterizes, for example, a similarity between each candidate molecule 12.i to each other candidate molecule 12.j. The virtual screening system 500 can repeat this procedure by assigning each candidate molecule 12.j as the reference molecule 13. This produces a respective pair-wise score 150.i.j for each pair of candidate molecules 12.i and 12.j in the set of candidate molecules 12.1 to 12.n. The virtual screening system 500 may then rank each of the pairs of candidate molecules 12.i and 12.j according to their pair-wise scores 150.i.j. More generally, the virtual screening system 500 can compare different pairs of candidate molecules 12.i and 12.j, determining a pair-wise score 150.i.j for each, in any of their possible permutations.

In more detail, the virtual screening system 500 prepares arespective input 115.a[i].b[i] for each candidate molecule 12.i in set of candidate molecules 12.1 to 12.n, which is thereafter processed by the free energy prediction system 100 to estimate the respective free energy difference 140.a[i]:b[i]. The input 115.a[i].b[i] specifies: (a) a set of thermodynamic parameters 112.a[i] of a first molecular system 110.a[i]; and (b) a set of thermodynamic parameters 112.b of a second molecular system 110.b[j]. Here, the virtual screening 500 and free energy prediction 100 systems implement a dual topology approach 300B as described above with reference to FIG. 3B, such that the free energy difference 140.a[i]:b[i] is a RBFED between the candidate 12.i and reference 13 molecules to the target molecular structure 10. However, the virtual screening 500 and free energy prediction 100 systems can implement any of the distinct 200A, single 200B, common 300A, or dual 300B topology approaches described above with reference to FIGS. 2A-3B.

In this implementation of the dual topology approach 300B, the first molecular system 110.a[i] includes the candidate molecule 12.i, the reference molecule 13, the target molecular structure 10, and a solvent 14, where the candidate molecule 12.i is positioned outside the binding site 11 of the target molecular structure 10, and the reference molecule 13 is positioned inside the binding site 11. Similarly, the second molecular system 110.b[i] includes the candidate molecule 12.i, the reference molecule 13, the target molecular structure 10, and the solvent 14, where the candidate molecule 12.i is positioned inside the binding site 11 of the target molecular structure 10, and the reference molecule 13 is positioned outside the binding site 11.

The free energy prediction system 100 processes the input 115.a[i].b[i], as described above with reference to FIGS. 1A-1C, to estimate the RBFED 140.a[i]:b[i] between the first 110.a[i] and second 110.a[i] molecular systems. Subsequently, the virtual screening system 500 determines the score 150[i] for the candidate molecule 12.i based on the RBFED 140.a[i]:b[i], or based on an ABFED between the candidate molecule 12.i and the target molecular structure 10 after adding the ABFED of the reference molecule 13 to the RBFED 140.a[i]:b[i] of the candidate molecule 12[i]. Alternatively, or in addition, the virtual screening system 500 can use the ABFED of the reference molecule 13 to solve for the ABFEDs of each of the candidate molecules 12.1 to 12.n via regression (e.g., linear or multiple regression) or other optimization models, e.g., to find the most likely ABFEDs of the candidate molecules 12.1 to 12.n from their RBFEDs 140. The virtual screening system 500 may then compute the disassociation constant (e.g., binding affinity) from the ABFED of the candidate molecule 12.i and use it directly as the score 150.i. That is, the virtual screening system 500 computes the score 150.i as

$${K}_{D} = exp ( - \frac{Δ {F}_{Bind}^{0}}{RT} ) .$$

Upon preforming this above procedure for each candidate molecule 12.i in the set of candidate molecules 12.1 to 12.n, or between pairs of candidate molecules 12.i and 12.j in any of the possible permutations, the virtual screening system 500 can rank the set of candidate molecules 12.1 to 12.n according to their scores 150.1 to 150.n and then select a subset of the candidate molecules 12.1 to 12.n that includes one or more of the candidate molecules 12*.k with the highest respective scores 150, e.g., corresponding to the highest binding affinities to the target molecular structure 10. After identifying the subset including the selected candidate molecule(s) 12*.k, a user of the virtual screening system 500 can physically synthesize (or otherwise obtain) each of the selected candidate molecules 12*.k and then perform one or more experiments on the physically synthesized candidate molecules to determine one or more of their physical properties. For example, the experiment(s) can include one or more of: microwave rotational spectroscopy, neutron diffraction, x-ray diffraction, electron spin resonance, nuclear magnetic resonance, or other chemical and biological experiments. Alternatively, or in addition, a user of the virtual screening system 500 may then design a therapeutic drug including each of the selected candidate molecule(s) 12*.k, e.g., in various concentrations, and thereafter administer the therapeutic drug in one or more clinical trials.

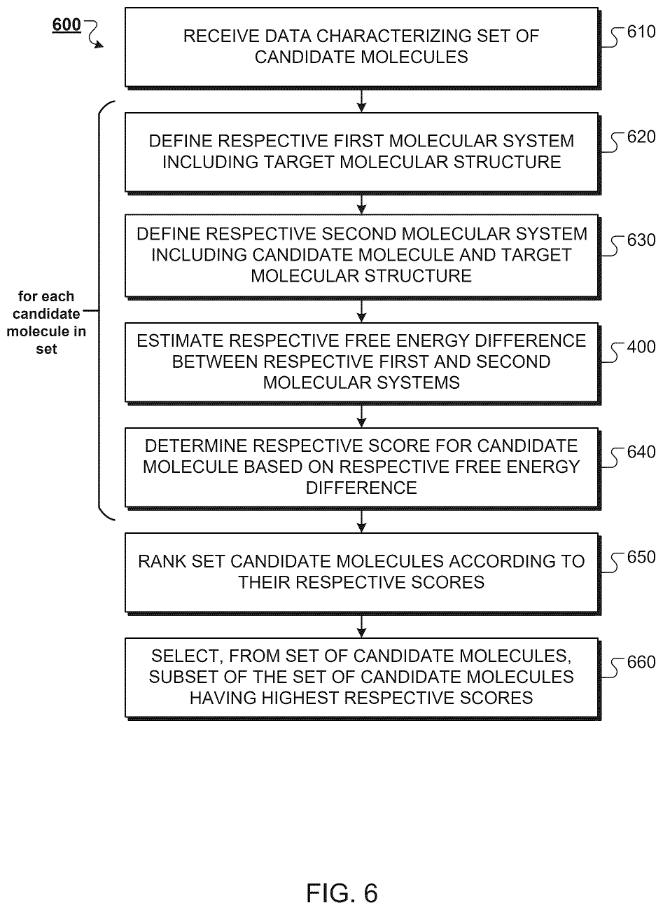

FIG. 6 is a flow diagram of an example process 600 for virtually screening a set of candidate molecules 12.1 to 12.n against a target molecular structure 10. For convenience, the process 600 will be described as being performed by a system of one or more computers located in one or more locations. For example, a virtual screening system, e.g., the virtual screening system 500 of FIG. 6, appropriately programmed in accordance with this specification, can perform the process 600.

The virtual screening system 500 receives data defining the set of candidate molecules 12.1 to 12.n (610).

For each candidate molecule 12.i in the set of candidate molecules 12.1 to 12.n, the virtual screening system 500 performs operations (620)-(640):

The virtual screening system 500 defines a respective first molecular system 110.a[i] including the target molecular structure 10 (620).

The virtual screening system 500 defines a respective second molecular system 110.b[i] including the candidate molecule 12.i and the target molecular structure 10 (630).

The virtual screening system 500 estimates, using the free energy prediction system 100, a respective free energy difference 140.a[i]:b[i] between the respective first 110.a[i] and second 110.b[i] molecular systems (400).

The virtual screening system 500 determines a respective score 150.a[i]:b[i] for the candidate molecule 12.i based on the respective free energy difference 140.a[i]:b[i] between the respective first 110.a[i] and second 110.b[i] molecular systems (640).

The virtual screening system 500 ranks the set of candidate molecules 12.1 to 12.n according to their respective scores 150.1 to 150.n (650).

The virtual screening system 500 selects, from the set of candidate molecules 12.1 to 12.n, a subset 12*.k of the set of candidate molecules 12.1 to 12.n having the highest respective scores 150*.k (660).

### SCOPE OF THE DISCLOSURE

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random-access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

### OTHER EMBODIMENTS

In addition to the embodiments of the attached claims and the embodiments described above, the following numbered embodiments are also innovative.
1. A method performed by one or more computers for estimating a free energy difference between: (a) a first molecular system, and (b) a second molecular system, the method comprising: receiving an input specifying a respective set of thermodynamic parameters of each of the first and second molecular systems; processing the input to generate a Hamiltonian of an alchemical system comprising the first and second molecular systems; parametrizing the Hamiltonian with an alchemical progress parameter that implements an alchemical transformation between the first and second molecular systems, wherein the alchemical transformation is specified by: a monotonic sequence of points including: (a) a first endpoint that parametrizes the Hamiltonian of the alchemical system as a Hamiltonian of the first molecular system; (b) a second endpoint that parametrizes the Hamiltonian of the alchemical system as a Hamiltonian of the second molecular system; and (c) one or more interior points that each parametrize the Hamiltonian of the alchemical system as a Hamiltonian of a respective intermediate molecular system; and for each contiguous pair of points in the monotonic sequence of points, a respective alchemical path connecting the pair of points; computing, via non-equilibrium switching along each alchemical path in the alchemical transformation, a respective free energy difference estimator between the corresponding pair of molecular systems connected by the alchemical path; and combining each of the free energy difference estimators to estimate the free energy difference between the first and second molecular systems.
2. The method of embodiment 1, wherein computing, via non-equilibrium switching along each alchemical path in the alchemical transformation, the respective free energy difference estimator between the corresponding pair of molecular systems connected by the alchemical path comprises, for a first of the pair of molecular systems: obtaining an ensemble of microstates of the first of the pair of molecular systems; specifying a forward switching rate at which the alchemical progress parameter traverses the alchemical path in a forward direction; and for each microstate in the ensemble of microstates: evolving the microstate under the Hamiltonian of the alchemical system while the alchemical progress parameter traverses the alchemical path at the forward switching rate; and evaluating a respective non-equilibrium work performed on the microstate during the evolving.
3. The method of embodiment 2, wherein computing, via non-equilibrium switching along each alchemical path in the alchemical transformation, the respective free energy difference estimator between the corresponding pair of molecular systems connected by the alchemical path further comprises, for the first of the pair of molecular systems: averaging, over the ensemble of microstates of the first of the pair of molecular systems, an exponential of the respective non-equilibrium work performed on each microstate in the ensemble to generate an exponential of the free energy difference estimator; and computing a logarithm of the exponential of the free energy difference estimator to obtain the free energy difference estimator between the pair of molecular systems.
4. The method of embodiment 2, wherein computing, via non-equilibrium switching along each alchemical path in the alchemical transformation, the respective free energy difference estimator between the corresponding pair of molecular systems connected by the alchemical path further comprises, for a second of the pair of molecular systems: obtaining an ensemble of microstates of the second of the pair of molecular systems; specifying a reverse switching rate at which the alchemical progress parameter traverses the alchemical path in a reverse direction; and for each microstate in the ensemble of microstates: evolving the microstate under the Hamiltonian of the alchemical system while the alchemical progress parameter traverses the alchemical path at the reverse switching rate; and evaluating a respective non-equilibrium work performed on the microstate during the evolving.
5. The method of embodiment 4, wherein computing, via non-equilibrium switching along each alchemical path in the alchemical transformation, the respective free energy difference estimator between the corresponding pair of molecular systems connected by the alchemical path further comprises, for the second of the pair of molecular systems: averaging, over the ensemble of microstates of the second of the pair of molecular systems, an exponential of the non-equilibrium work performed on each microstate in the ensemble to generate an exponential of a negative of the free energy difference estimator; computing a logarithm of the exponential of the negative of the free energy difference estimator to obtain the negative of the free energy difference estimator; and negating the negative of the free energy difference estimator to obtain the free energy difference estimator between the pair of molecular systems.
6. The method of embodiment 4, wherein computing, via non-equilibrium switching along each alchemical path in the alchemical transformation, the respective free energy difference estimator between the corresponding pair of molecular systems connected by the alchemical path further comprises: for each of the pair of molecular systems, generating, from the respective non-equilibrium work performed on each microstate in the respective ensemble of microstates of the molecular system, a respective probability distribution of work performed on the molecular system; determining, from the respective probability distributions of work performed on the pair of molecular systems, a particular value of work that has an equal likelihood of being performed on each of the pair of molecular systems; and identifying the particular value of work as the free energy difference estimator between the pair of molecular systems.
7. The method of embodiment 6, wherein, for each of the pair of molecular systems, generating, from the respective non-equilibrium work performed on each microstate in the respective ensemble of microstates of the molecular system, the respective probability distribution of work performed on the molecular system comprises: averaging, over the ensemble of microstates of the molecular system, a delta function of the respective non-equilibrium work performed on each microstate in the ensemble to generate the probability distribution of work performed on the molecular system.
8. The method of embodiment 7, wherein the delta function is approximated as a Gaussian function or a Lorentzian function.
9. The method of any one of embodiments 2-8, further comprising: generating the respective ensemble of microstates of each of the first molecular system, second molecular system, and one or more intermediate molecular systems using the respective Hamiltonian of the molecular system.
10. The method of embodiment 9, wherein one or more of the respective ensembles of microstates of the first molecular system, second molecular system, and one or more intermediate molecular systems is a non-equilibrium ensemble.
11. The method of embodiment 10, wherein each of the respective ensembles of microstates of the first molecular system, second molecular system, and one or more intermediate molecular systems is a non-equilibrium ensemble.
12. The method of any one of embodiments 9-11, wherein generating the respective ensemble of microstates of each of the first molecular system, second molecular system, and one or more intermediate molecular systems using the respective Hamiltonian of the molecular system comprises, for each of the first molecular system, second molecular system, and one or more intermediate molecular systems: performing one or more molecular simulations, each derived from the Hamiltonian of the molecular system, to generate the ensemble of microstates of the molecular system.
13. The method of embodiment 12, wherein each of the one or more molecular simulations is a Molecular Dynamics simulation or a Monte Carlo molecular simulation.
14. The method of any one of embodiments 12-13, wherein each of the one or more molecular simulations is performed with one or more enhanced sampling techniques.
15. The method of embodiment 14, wherein the one or more enhanced sampling techniques includes an importance sampling method, a generalized ensemble method, or both.
16. The method of any one of embodiments 1-15, wherein the free energy difference between the first and second molecular systems is a Helmholtz free energy difference.
17. The method of any one of embodiments 1-15, wherein the free energy difference between the first and second molecular systems is a Gibbs free energy difference.
18. The method of any one of embodiments 1-17, wherein the respective set of thermodynamic parameters of each of the first and second molecular systems comprises: a common temperature of the first and second molecular systems; a total number of molecular entities in the first or second molecular system; and a respective number of molecular degrees of freedom of each molecular entity in the first or second molecular system.
19. The method of embodiment 18, wherein the respective set of thermodynamic parameters of each of the first and second molecular systems further comprises: a respective mass of each molecular entity in the first or second molecular system; and a set of force field parameters of an interatomic potential energy between each molecular entity in the first or second molecular system.
20. The method of any one of embodiments 18-19, wherein each molecular entity in the first and second molecular systems is an atom.
21. The method of any one of embodiments 1-20, wherein: the Hamiltonian of the alchemical system comprises a linear combination of the respective Hamiltonians of the first and second molecular systems, and each coefficient of the linear combination is parametrized by the alchemical progress parameter.
22. The method of any one of embodiments 1-21, wherein the Hamiltonian of the alchemical system comprises: a kinetic energy of the alchemical system; and an alchemical potential energy parametrized by the alchemical progress parameter.
23. The method of any one of embodiments 1-22, wherein: each of the first and second molecular systems is a given molecular system in a respective first and second pose that are related by a coordinate transformation, and the free energy difference between the first and second molecular systems is a free energy difference between the first and second poses of the given molecular system.
24. The method of embodiment 23, wherein the given molecular system comprises a plurality of molecules.
25. The method of embodiment 24, wherein the coordinate transformation comprises, for a subset of the plurality of molecules, a respective rigid translation and/or a respective rigid rotation of each molecule in the subset.
26. The method of embodiment 23, wherein the given molecular system comprises one or more guest molecules and a target molecular structure.
27. The method of embodiment 26, wherein the coordinate transformation comprises, for each of the one or more guest molecules, a respective rigid translation of the guest molecule relative to the target molecular structure.
28. The method of any one of embodiments 26-27, wherein the given molecular system further comprises a solvent having the one or more guest molecules and the target molecular structure suspended therein.
29. The method of any one of embodiments 26-28, wherein: the one or more guest molecules consists of one guest molecule, the first pose of the given molecular system positions the guest molecule outside a binding site of the target molecular structure, the second pose of the given molecular system positions the guest molecule inside the binding site of the target molecular structure, and the free energy difference between the first and second poses of the given molecular system is an absolute binding free energy difference between the guest molecule and the target molecular structure.
30. The method of any one of embodiments 26-28, wherein: the one or more guest molecules consists of two guest molecules, the first pose of the given molecular system positions: (i) a first of the two guest molecules outside a binding site of the target molecular structure, and (ii) a second of the two guest molecules inside the binding site of the target molecular structure, the second pose of the given molecular system positions: (i) the first of the two guest molecules inside the binding site of the target molecular structure, and (ii) the second of the two guest molecules outside the binding site of the target molecular structure, and the free energy difference between the first and second poses of the given molecular system is a relative binding free energy difference between the two guest molecules to the target molecular structure.
31. The method of embodiment 30, wherein the relative binding free energy difference between the two guest molecules to the target molecular structure comprises a difference between: (i) an absolute binding free energy difference between the first of the two guest molecules and the target molecular structure, and (ii) an absolute binding free energy difference between the second of the two guest molecules and the target molecular structure.
32. The method of any one of embodiments 30-31, wherein the two guest molecules are congeneric.
33. The method of any one of embodiments 26-32, wherein each of the one or more guest molecules is a ligand or a peptide, and the target molecular structure is a protein.
34. The method of any one of embodiments 26-32, wherein each of the one or more guest molecules is a nucleic acid binding protein, and the target molecular structure is a nucleic acid.
35. The method of embodiment 34, wherein the nucleic acid is a deoxyribonucleic acid or a ribonucleic acid.
36. A method for screening a plurality of candidate molecules against a target molecular structure, the method comprising: for each of the plurality of candidate molecules: defining a respective first molecular system comprising the target molecular structure; defining a respective second molecular system comprising the candidate molecule and the target molecular structure; estimating, using the method of any one of embodiments 1-35, a respective free energy difference between the respective first and second molecular systems; and determining a respective score for the candidate molecule based on the respective free energy difference between the respective first and second molecular systems; ranking the plurality of candidate molecules according to their respective scores; and selecting, from the plurality of candidate molecules, a subset of the plurality of candidate molecules having the highest respective scores.
37. The method of embodiment 36, wherein, for each of the plurality of candidate molecules, each of the respective first and second molecular systems further comprises a solvent.
38. The method of any one of embodiments 36-37, wherein, for each of the plurality of candidate molecules, the respective free energy difference between the respective first and second molecular systems is a respective absolute binding free energy difference between the candidate molecule and the target molecular structure.
39. The method of any one of embodiments 36-37, wherein, for each of the plurality of candidate molecules: the respective first molecular system further comprises a reference molecule, and the respective free energy difference between the respective first and second molecular systems is a respective relative binding free energy difference between the candidate and reference molecules to the target molecular structure.
40. The method of embodiment 39, wherein the reference molecule and the plurality of candidate molecules are congeneric.
41. The method of any one of embodiments 39-40, wherein, for each of the plurality of candidate molecules, determining the respective score for the candidate molecule based on the respective relative binding free energy difference between the candidate and reference molecules to the target molecular structure comprises: adding, to the respective relative binding free energy difference, an absolute binding free energy difference between the reference molecule and the target molecular structure to obtain a respective absolute binding free energy difference between the candidate molecule and the target molecular structure; and determining the respective score for the candidate molecule based on the respective absolute binding free energy difference between the candidate molecule and the target molecular structure.
42. The method of any one of embodiments 36-41, further comprising: physically synthesizing each candidate molecule in the subset of candidate molecules.
43. The method of embodiment 42, further comprising: performing a set of experiments on each candidate molecule in the subset of candidate molecules to determine one or more respective physical properties of the candidate molecule.
44. The method of embodiment 43, wherein the set of experiments comprises one or more of: microwave rotational spectroscopy, neutron diffraction, x-ray diffraction, electron spin resonance, or nuclear magnetic resonance.
45. The method of any one of embodiments 36-44, wherein the target molecular structure is associated with one or more disease pathways of a disease, and the plurality of candidate molecules is screened against the target molecular structure for treating the disease.
46. The method of embodiment 45, further comprising: designing a therapeutic drug comprising each candidate molecule in the subset of candidate molecules.
47. The method of any one of embodiments 36-46, wherein each of the plurality of candidate molecules is a ligand or a peptide, and the target molecular structure is a protein.
48. The method of any one of embodiments 36-46, wherein each of the plurality of candidate molecules is a nucleic acid binding protein, and the target molecular structure is a nucleic acid.
49. A system, comprising: one or more computers; and one or more storage devices storing instructions that, when executed by the one or more computers, cause the one or more computers to perform the method of any one of embodiments 1-35.
50. One or more non-transitory computer storage media storing instructions that, when executed by one or more computers, cause the one or more computers to perform the method of any one of embodiments 1-35.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers for estimating a free energy difference between: (a) a first molecular system, and (b) a second molecular system, the method comprising:
receiving an input specifying a respective set of thermodynamic parameters of each of the first and second molecular systems;
processing the input to generate a Hamiltonian of an alchemical system comprising the first and second molecular systems;
parametrizing the Hamiltonian with an alchemical progress parameter that implements an alchemical transformation between the first and second molecular systems,
wherein the alchemical transformation is specified by:
a monotonic sequence of points including:
(a) a first endpoint that parametrizes the Hamiltonian of the alchemical system as a Hamiltonian of the first molecular system;
(b) a second endpoint that parametrizes the Hamiltonian of the alchemical system as a Hamiltonian of the second molecular system; and
(c) one or more interior points that each parametrize the Hamiltonian of the alchemical system as a Hamiltonian of a respective intermediate molecular system; and
for each contiguous pair of points in the monotonic sequence of points, a respective alchemical path connecting the pair of points;
computing, via non-equilibrium switching along each alchemical path in the alchemical transformation, a respective free energy difference estimator between the corresponding pair of molecular systems connected by the alchemical path; and
combining each of the free energy difference estimators to estimate the free energy difference between the first and second molecular systems.

2. The method of claim 1, wherein computing, via non-equilibrium switching along each alchemical path in the alchemical transformation, the respective free energy difference estimator between the corresponding pair of molecular systems connected by the alchemical path comprises, for a first of the pair of molecular systems:
obtaining an ensemble of microstates of the first of the pair of molecular systems;
specifying a forward switching rate at which the alchemical progress parameter traverses the alchemical path in a forward direction toward the second molecular system; and
for each microstate in the ensemble of microstates:
evolving the microstate under the Hamiltonian of the alchemical system while the alchemical progress parameter traverses the alchemical path at the forward switching rate; and
evaluating a respective non-equilibrium work performed on the microstate during the evolving.

3. The method of claim 2, wherein computing, via non-equilibrium switching along each alchemical path in the alchemical transformation, the respective free energy difference estimator between the corresponding pair of molecular systems connected by the alchemical path further comprises, for the first of the pair of molecular systems:
averaging, over the ensemble of microstates of the first of the pair of molecular systems, an exponential of the respective non-equilibrium work performed on each microstate in the ensemble to generate an exponential of the free energy difference estimator; and
computing a logarithm of the exponential of the free energy difference estimator to obtain the free energy difference estimator between the pair of molecular systems.

4. The method of claim 2, wherein computing, via non-equilibrium switching along each alchemical path in the alchemical transformation, the respective free energy difference estimator between the corresponding pair of molecular systems connected by the alchemical path further comprises, for a second of the pair of molecular systems:
obtaining an ensemble of microstates of the second of the pair of molecular systems;
specifying a reverse switching rate at which the alchemical progress parameter traverses the alchemical path in a reverse direction; and
for each microstate in the ensemble of microstates:
evolving the microstate under the Hamiltonian of the alchemical system while the alchemical progress parameter traverses the alchemical path at the reverse switching rate; and
evaluating a respective non-equilibrium work performed on the microstate during the evolving.

5. The method of claim 4, wherein computing, via non-equilibrium switching along each alchemical path in the alchemical transformation, the respective free energy difference estimator between the corresponding pair of molecular systems connected by the alchemical path further comprises, for the second of the pair of molecular systems:
averaging, over the ensemble of microstates of the second of the pair of molecular systems, an exponential of the non-equilibrium work performed on each microstate in the ensemble to generate an exponential of a negative of the free energy difference estimator;
computing a logarithm of the exponential of the negative of the free energy difference estimator to obtain the negative of the free energy difference estimator; and
negating the negative of the free energy difference estimator to obtain the free energy difference estimator between the pair of molecular systems.

6. The method of claim 4, wherein computing, via non-equilibrium switching along each alchemical path in the alchemical transformation, the respective free energy difference estimator between the corresponding pair of molecular systems connected by the alchemical path further comprises:
for each of the pair of molecular systems, generating, from the respective non-equilibrium work performed on each microstate in the respective ensemble of microstates of the molecular system, a respective probability distribution of work performed on the molecular system;
determining, from the respective probability distributions of work performed on the pair of molecular systems, a particular value of work that has an equal likelihood of being performed on each of the pair of molecular systems; and
identifying the particular value of work as the free energy difference estimator between the pair of molecular systems.

7. The method of claim 6, wherein, for each of the pair of molecular systems, generating, from the respective non-equilibrium work performed on each microstate in the respective ensemble of microstates of the molecular system, the respective probability distribution of work performed on the molecular system comprises:
averaging, over the ensemble of microstates of the molecular system, a delta function of the respective non-equilibrium work performed on each microstate in the ensemble to generate the probability distribution of work performed on the molecular system.

8. The method of claim 7, wherein the delta function is approximated as a Gaussian function or a Lorentzian function.

9. The method of claim 2, further comprising:
generating the respective ensemble of microstates of each of the first molecular system, second molecular system, and one or more intermediate molecular systems using the respective Hamiltonian of the molecular system.

10. The method of claim 9, wherein one or more of the respective ensembles of microstates of the first molecular system, second molecular system, and one or more intermediate molecular systems is a non-equilibrium ensemble.

11. The method of claim 10, wherein each of the respective ensembles of microstates of the first molecular system, second molecular system, and one or more intermediate molecular systems is a non-equilibrium ensemble.

12. The method of claim 9, wherein generating the respective ensemble of microstates of each of the first molecular system, second molecular system, and one or more intermediate molecular systems using the respective Hamiltonian of the molecular system comprises, for each of the first molecular system, second molecular system, and one or more intermediate molecular systems:
performing one or more molecular simulations, each derived from the Hamiltonian of the molecular system, to generate the ensemble of microstates of the molecular system.

13. The method of claim 12, wherein each of the one or more molecular simulations is a Molecular Dynamics simulation or a Monte Carlo molecular simulation.

14. The method of claim 12, wherein each the one or more molecular simulations is performed with one or more enhanced sampling techniques.

15. The method of claim 14, wherein the one or more enhanced sampling techniques includes an importance sampling method, a generalized ensemble method, or both.

16. The method of claim 1, wherein the free energy difference between the first and second molecular systems is a Helmholtz free energy difference.

17. The method of claim 1 wherein the free energy difference between the first and second molecular systems is a Gibbs free energy difference.

18. The method of claim 1, wherein the respective set of thermodynamic parameters of each of the first and second molecular systems comprises:
a common temperature of the first and second molecular systems;
a total number of molecular entities in the first or second molecular system; and
a respective number of molecular degrees of freedom of each molecular entity in the first or second molecular system.

19. A system, comprising:
one or more computers; and
one or more storage devices storing instructions that, when executed by the one or more computers, cause the one or more computers to perform operations for estimating a free energy difference between: (a) a first molecular system, and (b) a second molecular system, the operations comprising:
receiving an input specifying a respective set of thermodynamic parameters of each of the first and second molecular systems;
processing the input to generate a Hamiltonian of an alchemical system comprising the first and second molecular systems;
parametrizing the Hamiltonian with an alchemical progress parameter that implements an alchemical transformation between the first and second molecular systems,
wherein the alchemical transformation is specified by:
a monotonic sequence of points including:
(a) a first endpoint that parametrizes the Hamiltonian of the alchemical system as a Hamiltonian of the first molecular system;
(b) a second endpoint that parametrizes the Hamiltonian of the alchemical system as a Hamiltonian of the second molecular system; and
(c) one or more interior points that each parametrize the Hamiltonian of the alchemical system as a Hamiltonian of a respective intermediate molecular system; and
for each contiguous pair of points in the monotonic sequence of points, a respective alchemical path connecting the pair of points;
computing, via non-equilibrium switching along each alchemical path in the alchemical transformation, a respective free energy difference estimator between the corresponding pair of molecular systems connected by the alchemical path; and
combining each of the free energy difference estimators to estimate the free energy difference between the first and second molecular systems.

20. One or more non-transitory computer storage media storing instructions that, when executed by one or more computers, cause the one or more computers to perform operations for estimating a free energy difference between: (a) a first molecular system, and (b) a second molecular system, the operations comprising:
receiving an input specifying a respective set of thermodynamic parameters of each of the first and second molecular systems;
processing the input to generate a Hamiltonian of an alchemical system comprising the first and second molecular systems;
parametrizing the Hamiltonian with an alchemical progress parameter that implements an alchemical transformation between the first and second molecular systems,
wherein the alchemical transformation is specified by:
a monotonic sequence of points including:
(a) a first endpoint that parametrizes the Hamiltonian of the alchemical system as a Hamiltonian of the first molecular system;
(b) a second endpoint that parametrizes the Hamiltonian of the alchemical system as a Hamiltonian of the second molecular system; and
(c) one or more interior points that each parametrize the Hamiltonian of the alchemical system as a Hamiltonian of a respective intermediate molecular system; and
for each contiguous pair of points in the monotonic sequence of points, a respective alchemical path connecting the pair of points;
computing, via non-equilibrium switching along each alchemical path in the alchemical transformation, a respective free energy difference estimator between the corresponding pair of molecular systems connected by the alchemical path; and
combining each of the free energy difference estimators to estimate the free energy difference between the first and second molecular systems.
